(19) United States
(12) Patent Application Publication
(10) Pub. No.: US 2018/0092947 A1
(43) Pub. Date: Apr. 5, 2018

(54) L-MYC Expression Maintains Self Renewal and Prolongs Multipotency of Primary Human Neural Stem Cells

(71) Applicant: Margarita Gutova, La Cresenta, CA (US)

(72) Inventor: Margarita Gutova, La Cresenta, CA (US)

(21) Appl. No.: 15/679,463

(22) Filed: Aug. 17, 2017

Related U.S. Application Data
(60) Provisional application No. 62/376,822, filed on Aug. 18, 2016.

(51) Int. Cl. A61K 35/30; C07K 14/82; C12N 5/0797; C12N 15/86; A61K 45/06

(52) U.S. Cl. CPC A61K 35/30; C07K 14/82; C12N 5/0623; C12N 2500/90; A61K 45/06; C12N 2740/15043; C12N 15/86

(57) ABSTRACT

The invention provides isolated neural or mesenchymal stem cell populations which are genetically modified with an exogenous nucleic acid sequence encoding for a L-myc gene or its variant, wherein the L-myc gene or its variant is necessary and sufficient to immortalize the isolated neural or mesenchymal stem cell population.

This patent application claims the benefit of provisional patent application, U.S. Ser. No. 62/376,822, filed Aug. 18, 2016, the contents of which are herein incorporated by reference in its entirety into the present patent application.

Throughout this application various publications are referenced. The disclosures of these publications in their entireties are hereby incorporated by reference into this application in order to more fully describe the state of the art to which this invention pertains.

### BACKGROUND OF THE INVENTION

Despite decades of research, treatments for patients with diseased or damaged regions of the central nervous system (CNS) remain palliative at best (Pathan et al., 2009). Cell-based therapies are emerging as a novel and potentially powerful approach for treatment of CNS pathologies, and multipotent neural stem cells (NSCs) in particular are an attractive cell type for use in CNS therapies. Recent pre-clinical proof-of-concept studies have demonstrated the potential of NSC-based treatments for disorders requiring neural cell replacement (Begum et al., 2015), protection from external insult (Umeda et al., 2016), antibody production (Kanojia et al., 2015), and targeted delivery of therapeutic agents (Aboody et al., 2013), including prodrug-activating enzymes (Metz et al., 2013).

Allogeneic donor cells remain an attractive possibility if an appropriate source can be identified. Although the self-renewing NSCs present in developing brain tissue could be used as a renewable cell population, culture conditions have yet to be identified that reproducibly permit continuous propagation of primary NSCs. One common approach is to expand NSC pools by repeated subculture of polyclonal neurospheres. However, progressive passages lead to decreased capacity for cellular self-renewal, decreased differentiation potential, and increased accumulation of chromosomal and functional instabilities (Reynolds and Weiss, 1992; Kallos and Behie, 1999; Nakagawa et al., 2008). Thus a new source of primary tissue must be obtained for each production cycle, which makes process scale-up, regulatory approval, and clinical translation substantially more difficult and costly. A more practical approach has been to generate stable, immortal NSC lines by retroviral transduction of a MYC gene into early gestational NSC pools (Kim et al., 2008). These MYC immortalized NSC lines retain their self-renewal capabilities for substantial numbers of passages (>50 as opposed to 5-6 passages for non-immortalized NSCs), which exponentially increases the number of NSCs available for use in potential therapies (Kim, 2004).

There are have been safety-related concerns, as yet unrealized, related to MYC-based NSC immortalization, including the risk that a MYC transgene could render the NSC line tumorigenic upon transplantation (Nakagawa et al., 2010). However, the clonal v-MYC immortalized human NSC line, HB1.F3.CD21 has shown chromosomal and functional stability over multiple passages and GMP scale-up (Aboody et al., 2013), and demonstrated clinical safety and non-tumorigenicity in patients with recurrent glioblastoma (NCT02015819). Another NSC line (CTX0E03) was established in 2006 using the c-MYC gene commonly used in generation of induced pluripotent stem cells (Pollock et al., 2006; Nakagawa and Yamanaka, 2010; Hicks et al., 2013). In this case, to ensure that c-MYC expression could be controlled upon transplantation, a conditional technology was used to enable suppression of c-MYC via systemic tamoxifen administration if necessary (Pollock et al., 2006).

These two MYC-immortalized NSC lines exhibit properties that cause them to differ in their utility for treating diseases. Specifically, HB1.F3.CD21 NSCs exhibit superior tumor tropism but more limited differentiation capacity as compared to CTX0E03 NSCs. These cells secrete bioactive factors that are useful for promoting host neuronal plasticity or vascular growth into damaged brain areas and have demonstrated clinical efficacy in stroke patients (Stroemer et al., 2009). The reasons for these differences are unknown, but may arise from differences in the initial progenitor pools chosen (gestational age), specifics of culture and transduction conditions, and chromosomal insertion points.

We have developed a protocol for producing and characterizing new MYC-immortalized NSC lines that can be propagated up to at least passage 50. This protocol incorporates two recent technological advances that should further improve safety profiles relative to older MYC-immortalized NSC lines: (1) culture and freezing conditions that exclusively use human-derived supplements, and, most importantly, (2) transduction with L-MYC to reduce the risk of transformation (Nakagawa et al., 2008). L-MYC has significantly lower transformation activity in cultured cells than the other MYC members (Oster et al., 2003); and only a small number of human cancers have been associated with the aberrant expression of L-MYC (Nakagawa et al., 2010).

Here we describe generation of the first L-MYC immortalized human NSC line derived from human fetal brain, for brevity referred to as LM-NSC008. We find that the LM-NSC008 line is chromosomally normal, and over-expresses several stem cell-associated genes. LM-NSC008 cells proliferate up to passage 50 in vitro, as compared with the parental untransduced NSC008 cells, and can be expanded under both suspension and adherent culture conditions (an advantage for eventual clinical scale-up). The LM-NSC008 stem cell line is non-tumorigenic when injected to non-tumor bearing mouse brain for up to 9 months. Further, these cells exhibit tropism towards brain tumors and traumatic brain injury (TBI) sites and differentiate into neural, astrocytic and oligodendroglial lineages, as do normal non-immortalized NSCs.

We further characterized the stability of LM-NSC008s in in vitro passages (up to 50) and long-term fate of LM-NSC008 NSCs in non-tumor-bearing brain for up to 9 months. We also performed computational analyses of tissue structure, orientation, and anisotropy to characterize preferential routes of NSC migration, which may be used to predict routes of migration and spatial distribution of LM-NSC008 cells within the brain. Once developed into a ‘clinical-grade’ stem cell line, LM-NSC008 may find widespread use in stem cell-mediated therapies for CNS pathologies.

### SUMMARY OF THE INVENTION

Preclinical studies indicate that neural stem cells (NSCs) can limit or reverse central nervous system (CNS) damage through direct cell replacement, promotion of regeneration, or delivery of therapeutic agents. Immortalized NSC lines are in growing demand due to the inherent limitations of adult patient-derived NSCs, including availability, expandability, potential for genetic modifications, and costs. Here, we describe the generation and characterization of a new human fetal NSC line, immortalized by transduction with L-MYC (LM-NSC008) that in vitro displays both self-renewal and multipotent differentiation into neurons, oligodendrocytes and astrocytes. These LM-NSC008 cells were non-tumorigenic in vivo, and migrated to orthotopic glioma xenografts in immunodeficient mice. When administered intranasally, LM-NSC008 distributed specifically to sites of traumatic brain injury (TBI). We demonstrated the long-term stability and lack of tumorigenicity of an L-MYC immortalized human NSC line (LM-NSC008) in vitro and in vivo. We also computationally analyzed LM-NSC008s spatial distributions and quantified NSC migration characteristics in relation to intrinsic brain structure as assessed through brightfield imaging. These data support the therapeutic use of allogeneic LM-NSC008s in traumatic brain injury and other CNS pathologies. These data support the therapeutic development of immortalized LM-NSC008 cells for allogeneic use in TBI and other CNS diseases.

### BRIEF DESCRIPTION OF THE FIGURES

FIGS. 1A-E. Visualization of primary NSCs in culture

(A) Primary NSCs (NSC008) were isolated from the brain of a 10-week-old fetus and cultured in flasks (passage 5). Primary NSCs grew as neurospheres in suspension. Scale bar, 100 μm. (B) Primary NSCs were transduced with a retroviral virus expressing the L-MYC gene. NSC.Lmyc cells (LM-NSC008) were cultured under the same conditions as untransduced primary NSCs. LM-NSC008 cells preferentially grew as a monolayer. Scale bar, 100 μm. (C) PCR analysis of genomic DNA derived from LM-NSC008 cells. The experiment was repeated three times. (D and E) Karyotype analysis of the LM-NSC008 cell line at passage 2 (D) and passage 10 (E).

FIGS. 2A-H. Growth kinetics and migration of primary NSC008 and LM-NSC008 cells in culture

(A) Proliferation of primary NSC008 and LM-NSC008 cells (passage 6) grown for 10 days in culture media (serum-free media containing FGF and EGF). Mean values±SD of two independent experiments in triplicate measurements are shown. (B) Flow cytometry analysis of NSC008 and LM-NSC008 expressing stem cell markers. Mean values±SD of four independent experiments in triplicates are shown. (C) Tropism of NSC008 and LM-NSC008 cells to conditioned media from U87 human glioma cells as assessed by Boyden chamber migration assay. Mean values±SD of three independent experiments in triplicate measurements are shown. (D) Dose response of U251 human glioma cells to CPT-11 and to CPT-11 in the presence of hCElm6 expressed by LM-NSC008 cells. Mean values±SD of two independent experiments in quadruplicates are shown. RLU, relative light unit. (E-H) Immunocytochemical staining of LM-NSC008 cells grown for 10 days in differentiation media. Cells were stained for neuronal class III b-TUBULIN (E), the astrocyte markers SOX9 (F) and GFAP (G) (inset STEM123), and oligodendrocyte marker 04 (H). Scale bars, 100 μm.

FIGS. 3A-G. Tumor-Specific Migration of LM-NSC008 Cells from Contralateral Hemisphere In Vivo (A, B, D, E) U251 human glioma xenografts were established in adult NSG mice. Consecutive horizontal brain sections from a tumorbearing mouse that received an intracranial (contralateral to the tumor) injection of LM-NSC008 cells labeled with feraheme. (A) Immunohistochemical staining of adjacent brain sections with EGFP antibodies to visualize tumor xenografts (scale bar, 1000 μm). (D) Enlargement of tumor area shown as inset in (A) (scale bar, 100 μm). (B) Prussian blue staining of axial brain sections, feraheme-labeled NSCs (blue) (scale bar, 1000 μm). (E) Higher-magnification of tumor site shown as inset in (B) with blue LM-NSC008 cells (scale bar, 100 μm). (C and F) Three-dimensional reconstruction of tumors (green) and LM-NSC008 cells (red) (n=6). (F) 3D enlargement of tumor area shown as inset in (C). (G) A summary of 3D reconstructions of tumors and LM-NSC008 cells at the tumor site. Scale bars 1,000 μm (A-C) and 100 μm (D-F). Asterisk indicates a mouse number.

FIGS. 4A-G. Distribution of LM-NSC008 Cells to TBI Sites (A) Prussian blue staining of brain section with TBI, demonstrating iron-labeled LM-NSC008 cells (blue) distributed throughout injury site. Scale bar, 500 μm. (B) Magnified area of TBI described in (A). Scale bar, 100 μm. (C) 3D reconstruction of LM-NSC008 cells distributed in the vicinity of TBI site. Scale bar, 150 μm. (D) Prussian blue staining of sham-injured brain section demonstrating rare (10-20) LM-NSC008 cells occasionally visualized around sham surgery sites. Scale bar, 500 μm. (E) Magnified area of injury site described in (D) Scale bar, 100 μm. (F) 3D reconstruction of LM-NSC008 cell distribution in sham-injured brain. Scale bar, 150 μm. (G) Quantification of average numbers of HB1.F3.CD and LM-NSC008 cells at TBI and sham injury sites (n=5). The arrows in (B, C, E, F) indicate LM-NSC008 cells in the vicinity of TBI or sham injury site. Statistical analysis of the treated and control groups was performed using one-way ANOVA. Tukey's adjusted p values are reported for pairwise comparisons of migration of hNSCs to TBI and sham injury sites.

FIGS. 5A-L. LM-NSC008 cell fate in non-tumor bearing brain. NSG mice were injected intracerebrally with LM-NSC008 cells (right frontal lobe) and euthanized 1 week (A, D, G), 4 weeks (B, E, H), and 12 weeks (C, F, I) (n=6) after injection. (A-C) Immunohistochemical (IHC) detection of apoptotic NSCs by using TUNEL staining. (D-F) IHC detection of the human-specific cell proliferation marker Ki-67. (G-I) IHC of mouse brain tissue using human-specific NESTIN antibodies. (J-L) z-Axis projection of all Ki-67- and hNESTIN-stained sections. NESTIN staining pseudocolored green and Ki-67 stained red.

FIGS. 6A-B. Summary of Up- and Downregulated Genes within Samples Analyzed (A) Untransduced NSC008 at passage 2 were compared with NSC008 at passage 9, L-MYC transduced cells (LM-NSC008) were compared at passages 2 versus 12, and NSC008 and LM-NSC008 cells were compared with differentiated LM-NSC008 cells. (B) Heatmap showing upregulated (red) and downregulated (green) genes.

FIGS. 7A-D. In vitro stability of LM-NSC008 cells. (A) PCR analysis of genomic DNA derived from LM-NSC008 cells at every 5th passage in vitro (p5-p50). Controls were: (i) L-MYC plasmid, (ii) DNA derived from untransduced NSC008 cells, (iii) no DNA template. The values for L-MYC band intensities from two experiments were compared using one-way ANOVA and showed no statistical difference among the bands (P=0.058); (B) Quantitative RT-PCR analysis for L-MYC gene expression (using mRNA as a template): fold change of L-MYC expression relative to GAPDH (mean±SD, n=3). Statistical analysis of an overall level of L-MYC gene expression within LM-NSC008 cells over 50 passages (as one group) in comparison to untransduced NSC008 mRNA levels showed statistical significance (P-value 0.001 using one-way ANOVA) in vitro; (C) Protein expression profile of LM-NSC008 cells: 1) CM from LM-NSC008 cells was collected; 2) LM-NSC008 cells were detached from the flask, pelleted and spun down to get the cell pellet. Cells were lysed and culture media (CM) were used for Protein Arrays (p5 and 45): cell lysates (left panels), CM (right panels) and key for cytokine array (below). (D) Key for cytokine antibody array V (RayBiotech).

FIGS. 8A-H. (A-D) IHC staining of mouse brain sections for hu-Nestin antibody. LM-NSC008 cells (brown) 6 months (A, B) and 9 months (E, F) post injection. (C, D, E, H) Three dimensional (3D) reconstruction of mouse brains using Reconstruct software (SynapseWeb, version 1.1). LM-NSC008 cells (green) 6 months (C, D) and 9 months (G, H) post injection. Scale bars 10001m.

FIGS. 9A-F. Distribution of LM-NSC008 cells. (A) Distance of LM-NSC008 cell clusters from the injection site at 3, 6, and 9 months post injection. Distances were calculated as Euclidean distances in the 2-dimensional plane. (B, C) Cumulative probability distributions of distance of LM-NSC008 clusters from the injection site in white and grey matter at 3, 6 and 9 months post injection indicating the migration of NSCs from the injection site. (D) Percentage of LM-NSC008 cells identified in the white matter at 3, 6, and 9 months post injection. (E, F) Cumulative probability distribution of distance from white matter/grey matter (WM/GM) interface where LM-NSC008 cells are seen to increasingly aggregate over time.

FIGS. 10A-G. Computational analysis of LM-NSC008 and tissue orientation. (A) Nestin stained NSCs through a cross-section of naïve mouse brain 9 months after IC injection of NSCs. (B) DiI myelin staining defining regions of white matter. (C) Relative tissue anisotropy (coherence) generated by the OrientationJ plugin to FIJI with 1 pixel Gaussian kernel. (D) Orientation vectors of white matter overlaid on the coherence image. The angle θ_{WM} is the orientation of the white matter. (E) NSC density overlaid on the DiI defined white matter boundaries, excluding the injection site. The green curve through the NSC cluster in corpus callosum shows the curve fit through the NSC cluster. The angle θ_{NSC} is the orientation angle of the NSC cluster. (F) Correlation between orientation of NSC clusters (θ_{NSC}) and the white matter (θ_{WM}) weighted by cluster size. (G) Stochastic simulations of 500 NSC migration paths overlaid on the coherence map evaluated using 5 pixel Gaussian kernel. Preferential migration along the corpus callosum is evident. Inset shows the paths near the seed initialization region contrasting the directional motility in the white and grey matter.

FIGS. 11A-G. (A) Three Dimensional (3D) mouse brain model generated using Reconstruct software (SynapseWeb, version 1.1). Brain histological sections, stained with STEM123, were imaged, aligned and used for 3D model. Mouse brain outlined (orange) and LM-NSC008 expressing eGFP (green). Scale bars 1000 μm. (B) Bright-field image of LM-NSC008s stained with STEM123 and contrastained with hematoxylin. Arrows showing LM-NSC008s (brown) in corpus callosum. Scale bars 200 μm; (C-G) IHC staining for huNestin (green)/TUJ1 (red) of mouse brain sections (insets). Counterstaining with DAPI (blue) to localize nuclei. White arrows showing LM-NSC008 cells double positive for huNestin (green)/TUJ1 (red). Scale bars 10 μm.

Table 1. DAVID Functional annotation analysis LM-NSC008 p2 vs. LM-NSC008 p12

Figure S1 A-C. In vitro tumorigenicity of neural stem cells.

Figure S2A-G. Proliferation and differentiation of LM-NSC008 cells in vitro.

Figure S3A-F. Immunocytochemistry for differentiation markers.

Figure S4A-F. L-myc staining of brain sections.

Figure S5. Human nestin expression based on 3D analysis of total volume of nestin positive cells.

Figure S6. Morphology of LM-NSC008 cells in culture. Images of LM-NSC008 cells in culture at passages 5, 10, and 45. Scale bars, 100 μM.

Table S1. DAVID Functional annotation analysis NSC008 p2 vs. NSC008 p9

Table S2. DAVID Functional annotation analysis of NSC008 and LM-NSC008 vs. differentiated LM-NSC008

Table S3. Gene expression level for SOX2, PAX6 and OCT4 genes.

Table S4. Copy Number Variation (CMV) detection analysis in LM-NSC008 cells

### DETAILED DESCRIPTION OF THE INVENTION

Compositions of the Invention

The invention provides an isolated neural or mesenchymal stem cell population which is genetically modified with an exogenous nucleic acid sequence encoding for a L-myc gene or its variant, wherein the L-myc gene or its variant is necessary and sufficient to immortalize the isolated neural or mesenchymal stem cell population.

In accordance with the practice of the invention, the L-myc gene is any of a human L-myc gene, bovine L-myc gene, porcine L-myc gene, murine L-myc gene, equine L-myc gene, canine L-myc gene, feline L-myc gene, simian L-myc gene, ovine L-myc gene, piscine L-myc gene or avian L-myc gene. In a preferred embodiment, the L-myc gene or its variant is a human L-myc gene.

In one embodiment, the isolated neural or mesenchymal stem cell population is not derived from an induced pluripotent stem cell population. In another embodiment, the stem cell population is genetically modified only with the L-myc gene. In another embodiment, the stem cell population is genetically modified with the L-myc gene, as the active agent necessary and sufficient to immortalize the isolated stem cell. In another embodiment, the stem cell population is genetically modified with the L-myc gene under the control of a constitutive promoter. In another embodiment, the stem cell population is genetically modified with the L-myc gene under the control of a constitutive promoter and enhancer. In another embodiment, the stem cell population is genetically modified with the L-myc gene under the control of a constitutive promoter, free of control by a regulatable or inducible promoter. In another embodiment, the stem cell population is genetically modified with the L-myc gene under the control of a constitutive promoter, free of control by a regulatable or inducible promoter and enhancer. In another embodiment, the stem cell population is genetically modified with the L-myc gene under the control of a regulatable or inducible promoter. In another embodiment, the stem cell population is genetically modified with the L-myc gene under the control of a regulatable or inducible promoter and enhancer. In one embodiment, the regulatable or inducible promoter or regulatable or inducible promoter and enhancer is controllable by a small molecule, temperature, metal, hormone or antibiotic. In one embodiment, the regulatable or inducible promoter or regulatable or inducible promoter and enhancer is controllable by manipulation of the environment of the NSC or its progenitor cell. In one embodiment, the regulatable or inducible promoter or regulatable or inducible promoter and enhancer permit upregulation or downregulation of the introduced L-myc gene expression. In one embodiment, the regulatable or inducible promoter or regulatable or inducible promoter and enhancer permit upregulation or downregulation of the introduced L-myc gene transcription. In one embodiment, the regulatable or inducible promoter or regulatable or inducible promoter and enhancer permit upregulation or downregulation of the introduced L-myc coding sequences. In yet another embodiment, the isolated neural or mesenchymal stem cell population is free of genetic modification with an exogenous nucleic acid sequence encoding for a member of Myc transcription factor family (c-myc, N-myc, v-myc) other than L-myc or its variant, an Octomer-binding protein (Oct) transcription factor (Oct-3/4), a Sox transcription factor (Sox1, Sox2, Sox3, Sox9, Sox15, and Sox18), a Klf transcription factor (Klf1, Klf2, Klf4, and Klf5), a Nanog transcription factor, a LIN28 RNA-binding protein, a Glis1 transcription factor, a short hairpin RNA targeting p53, or a combination thereof. For example, the member of Myc transcription factor family other than L-myc or its variant may be any of c-myc, N-myc and v-myc.

In accordance with the practice of the invention, the isolated neural or mesenchymal stem cell population may be obtained or is derived from a mammal. The mammal includes, but is not limited to, a mouse, a rat, a rabbit, a cat, a dog, a bovine, a goat, a pig, a horse, a sheep, a monkey, a chimpanzee, and a human. In a preferred embodiment, the cell population is from a human. In another specific embodiment, the mammal is a rat or rabbit. The yet another specific embodiment the mammal is a higher primate (e.g., a baboon or ape).

In further embodiment, the cell population is free of genetic modification with an exogenous nucleic acid sequence encoding for a reprogramming factor or immortalization factor other than L-myc gene or its variant. For example, the reprogramming factor or immortalization factor may confer increased self-renewal, promotes de-differentiation, and maintains pluripotency or multipotency, confers pluripotency or multipotency, or a combination thereof. Examples of suitable reprogramming factor or immortalization factor include, but are not limited to, any of Oct4, Sox2, Klf4, c-Myc, Lin28, Nanog, Sal14, Utf1, p53, p21, p16^{lnk4a}, GLIS1, TGF-β, MDM2, REM2, Cyclin D1, SV40 large T antigen, DOT1L, Cx43, MBD3, Sirt6, TCL1a, RARy, SNAIL, Lrh-1, RCOR2, miR367, LincRNA-ROR, miR302, miR766, miR200c, miR369, miR372, or Let7.

In one embodiment of the invention, greater than 90% of the neural stem cell population is positive for SOX2 and NESTIN neural stem cell markers. For example, up to 25% of the neural stem cell population may be positive for Oct-4. In another embodiment, the isolated neural stem cell population is positive for endogenous Notch 1 gene expression. In yet another embodiment, the neural stem cell population is negative for Olig1, Olig2 or Olig3 expression. Examples of Octomer-binding protein (Oct) transcription factor include any of Oct-3/4 and POU5F1. Examples of Sox transcription factor include any of Sox1, Sox2, Sox3, Sox9, Sox15, and Sox18. Examples of Klf transcription factor include any of Klf1, Klf2, Klf4, and Klf5.

In another embodiment of the invention, the isolated neural or mesenchymal stem cell population may be free of genetic modification with an exogenous nucleic acid sequence encoding for Oct-3/4, Sox2, Klf4, c-myc, N-myc, v-myc, Nanog, Lin28, Glis1, a short hairpin RNA targeting p53, miR-291, miR-294, miR-295 or a combination thereof.

Expression of the exogenous nucleic acid sequence encoding for a L-myc gene or its variant may confer immortalization of an isolated neural or mesenchymal stem cell population. In one embodiment, immortalization comprises increased self-renewal and maintenance of multipotency of a neural or mesenchymal stem cell population obtained from a subject. For example, increased self-renewal and maintenance of multipotency of a neural or mesenchymal stem cell population may be beyond 50 cell passages. In another example, increased self-renewal and maintenance of multipotency of a neural or mesenchymal stem cell population is at least 50 cell passages. In yet another example, increased self-renewal and maintenance of multipotency of a neural or mesenchymal stem cell population is beyond 50 cell passages. In a further example, the isolated neural or mesenchymal stem cell population can be cultured for at least about 50 passages without differentiating. In one embodiment, the cell population is cultured in vitro. For example, the cell population may be cultured as a monolayer. In one embodiment, the isolated neural or mesenchymal stem cell population has a cell population doubling time of approximately 30 to 36 hours. Further, the cell population may be cultured in a serum-free neural stem cell medium supplemented with basic fibroblast growth factor and epidermal growth factor.

In accordance with the practice of the invention, the isolated neural or mesenchymal stem cell population may be allogeneic or autologous. In one embodiment, the cell population is not derived from an isolated fibroblast or genetically modified fibroblast. In another embodiment, the cell population is not derived from reprogramming of a terminally differentiated cell.

In one embodiment of the invention, the isolated neural or mesenchymal stem cell population differentiates into neural cells. Further, in one embodiment, the neural cells so differentiated is positive for Class III 3-tubulin.

In another embodiment of the invention, the isolated neural or mesenchymal stem cell population differentiates into glial cells (positive for STEM123 and GFAP). Additionally, in one embodiment, the glial cells are oligodendrocytes. For example, the oligodendrocytes may be positive for oligodendrocyte marker O4. In yet another embodiment, the glial cells are astrocytes. For example, the astrocytes may be positive for SOX9 and GFAP markers.

In accordance with the practice of the invention, the neural or mesenchymal stem cell population may be differentiated in vitro. In an embodiment, the neural or mesenchymal stem cell population may be non-tumorigenic investigated up to 9 month in vivo. In one example, the non-tumorigenic neural or mesenchymal stem cell population is Ki-67, NESTIN or a combination of both. In another embodiment, the neural or mesenchymal stem cell population possesses proliferative and migratory properties through at least passage 50 in vitro. In yet another embodiment, the neural or mesenchymal stem cell population possesses migratory potential and tropism for a tumor site in vivo. For example, the tumor site in vivo may be intracranial. In another example, the tumor site in vivo may at a brain tissue. In another example, the tumor site in vivo may at a brain tissue. In yet another embodiment, the neural or mesenchymal stem cell population possesses migratory potential and tropism for a site of injury in vivo. In a further embodiment, the injury is central nervous system injury. For example, the central nervous system injury may be a traumatic brain injury. In another embodiment, the neural or mesenchymal stem cell population possesses intranasal and intracranial migratory potential in vivo.

In yet another embodiment, the neural or mesenchymal stem cell population possesses migratory potential and tropism, aggregating at white matter/grey matter interface. In yet another embodiment, the neural or mesenchymal stem cell population possesses migratory potential and tropism, aggreagating with white matter. In yet another embodiment, the neural or mesenchymal stem cell population possesses migratory potential and tropism, aligning with white matter. In yet another embodiment, the neural or mesenchymal stem cell population possesses migratory potential and tropism, aggreagating and aligning with white matter.

For example, the isolated neural or mesenchymal stem cell population may be used for brain tissue repair, brain tissue regeneration or a combination of both. In another embodiment, the isolated neural or mesenchymal stem cell population may be used for therapeutic drug delivery to a CNS tumor.

In one embodiment of the invention, the isolated neural or mesenchymal stem cell population maintains a normal diploid karyotype.

The invention further provides a defined cell population comprising a plurality of the isolated neural or mesenchymal stem cell population of the invention. In one embodiment, the defined cell population is homogenous. In another embodiment, the defined cell population is heterogeneous. In yet another embodiment, the defined cell population is clonal population.

The invention yet further provides a progeny cell of the isolated neural or mesenchymal stem cell population committed to develop into a neural cell. The invention yet further provides progeny cell of the isolated neural or mesenchymal stem cell population committed to develop into a glial cell. Additionally, the invention provides a progeny cell of the isolated neural or mesenchymal stem cell population committed to develop into a bone cell. The invention also provides a progeny cell of the isolated neural or mesenchymal stem cell population committed to develop into a cartilage cell. The invention also provides a progeny cell of the isolated neural or mesenchymal stem cell population committed to develop into a muscle cell. Further still, the invention provides a progeny cell of the isolated neural or mesenchymal stem cell population committed to develop into a fat cell.

Methods of the Invention

The invention additionally provides methods of producing an expandable neural or mesenchymal stem cell lines of the invention (see section “Compositions of the Invention”). In one embodiment, the method comprises (a) isolating neural or mesenchymal stem cells from a subject; (b) introducing an exogenous nucleic acid sequence encoding for a L-myc gene or its variant into the isolated neural or mesenchymal stem cells of (a), wherein the L-myc gene or its variant is necessary and sufficient to immortalize the isolated neural or mesenchymal stem cells so as to permit at least 50 passages without differentiating; and (c) culturing the cells of (b) under a condition permissive for self-renewal of neural or mesenchymal stem cells thereby, producing the expandable neural or mesenchymal stem cell line. In one embodiment, the cells in step (c) may be grown in a feeder-free chemically defined, serum-free medium.

For example, the neural stem cells of step (a) may be derived from brain. In one embodiment, the mesenchymal stem cells of step (a) are derived from bone marrow.

A “subject” is a vertebrate, preferably a mammal, more preferably a human. Mammals include, but are not limited to, farm animals (such as cows, sheep, and goats), sport animals, pets (such as cats, dogs and horses), primates (such as, monkeys, gorillas and chimpanzees), mice and rats.

An “effective amount” refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

In an embodiment of the invention, the exogenous nucleic acid comprises L-myc-coding sequence or L-myc variant-coding sequence, which is necessary and sufficient to immortalize the isolated neural or mesenchymal stem cells of the invention. For example, the human L-myc-coding sequence is disclosed in Nakagawa et al. (2010) PNAS USA, 107, 14152 which is incorporated herein. Further, in one embodiment, the exogenous nucleic acid additionally comprises a vector sequence operationally linked to L-myc-coding sequence or L-myc variant-coding sequence. In yet another embodiment, the exogenous nucleic acid may be stably propagated in neural or mesenchymal stem cells of the invention. In a further embodiment, the exogenous nucleic acid may be integrated into host chromosomal DNA or exists as an episomal DNA.

In accordance with the practice of the invention, in one embodiment, the vector is a derived from a DNA or RNA virus. Further, the vector may be a retroviral vector or lentiviral vector. Additionally, the exogenous nucleic acid sequence so introduced expresses L-myc protein or its variant. Further, the expression of L-myc protein or its variant is stable. In one embodiment, stable expression is confirmed by L-myc expression throughout different passages 5, 10, 15, 20, 25, 30, 35, 40, 45 and 50. In one embodiment, L-myc expression is constitutive. Constitutive expression of L-Myc is observed at least over 50 passages. L-Myc gene may be expressed using a retroviral transduction method, which, for example, inserts the L-myc gene into the genome and provides a stable expression of the gene.

The invention additionally provides a method of replenishing lost neural cells or repairing damaged neural cells in a subject comprising administering a cell of the invention to the subject under conditions sufficient for the cell to propagate, migrate, differentiate or a combination thereof, so as to provide a reservoir of neural stem cells, neural progenitor cells and neural cells to replace lost neural cells or repair damaged neural cells in the subject, thereby, replenishing lost neural cells or repairing damaged neural cells in the subject.

In one embodiment, the neural cells are neurons. In one embodiment, the neurons are dopaminergic neurons, GABAergic neurons, glutamatergic neurons, motor neurons or combination thereof. In one embodiment, the neural cells are glial cells. In one embodiment, the glial cells are oligodendrocytes, astrocytes or combination thereof. In one embodiment, the neural cells are neurons and glial cells.

The invention additionally provides a method of replenishing lost neuronal cells or repairing damaged neuronal cells in a subject comprising administering a cell of the invention to the subject under conditions sufficient for the cell to propagate, migrate, differentiate or a combination thereof, so as to provide a reservoir of neural stem cells, neural progenitor cells and neuronal cells to replace lost neuronal cells or repair damaged neuronal cells in the subject, thereby, replenishing lost neuronal cells or repairing damaged neuronal cells in the subject.

The invention additionally provides a method of replenishing lost neuronal cells or repairing damaged neuronal cells in a subject comprising administering a cell of the invention to the subject under conditions sufficient for the cell to propagate, migrate, differentiate or a combination thereof, so as to provide a reservoir of neural stem cells, neuronal progenitor cells and neuronal cells to replace lost neuronal cells or repair damaged neuronal cells in the subject, thereby, replenishing lost neuronal cells or repairing damaged neuronal cells in the subject.

Also, the invention provides a method of replenishing lost glial cells or repairing damaged glial cells in a subject comprising administering a cell of the invention to the subject under conditions sufficient for the cell to propagate, migrate, differentiate or a combination thereof, so as to provide a reservoir of neural stem cells, glial progenitor cells, astrocytes and oligodendrocytes to replace lost glial cells or repair damaged glial cells in the subject, thereby, replenishing lost glial cells or repairing damaged glial cells in the subject.

Also, the invention provides a method of replenishing lost astrocytes or repairing damaged astrocytes in a subject comprising administering a cell of the invention to the subject under conditions sufficient for the cell to propagate, migrate, differentiate or a combination thereof, so as to provide a reservoir of neural stem cells, glial progenitor cells and astrocytes to replace lost astrocytes or repair damaged astrocytes in the subject, thereby, replenishing lost astrocytes or repairing damaged astrocytes in the subject.

Also, the invention provides a method of replenishing lost oligodendrocytes or repairing damaged oligodendrocytes in a subject comprising administering a cell of the invention to the subject under conditions sufficient for the cell to propagate, migrate, differentiate or a combination thereof, so as to provide a reservoir of neural stem cells, glial progenitor cells and oligodendrocytes to replace lost oligodendrocytes or repair damaged oligodendrocytes in the subject, thereby, replenishing lost oligodendrocytes or repairing damaged oligodendrocytes in the subject.

The invention additionally provides a method of replenishing lost neural progenitor cells or repairing damaged neural progenitor cells in a subject comprising administering a cell of the invention to the subject under conditions sufficient for the cell to propagate, migrate, differentiate or a combination thereof, so as to provide a reservoir of neural stem cells and neural progenitor cells to replace lost neural progenitor cells or repair damaged neural progenitor cells in the subject, thereby, replenishing lost neural progenitor cells or repairing damaged neural progenitor cells in the subject.

The invention additionally provides a method of replenishing lost neural stem cells in a subject comprising administering a cell of the invention to the subject under conditions sufficient for the cell to propagate, migrate, differentiate or a combination thereof, so as to provide a reservoir of neural stem cells to replace lost neural stem cells in the subject, thereby, replenishing lost neural stem cells in the subject. In one embodiment, migration is to a neural stem cell niche in the subject. In one embodiment, migration is to a neural stem cell niche deficient in stem cells in the subject.

The invention additionally provides a method of repairing damaged neural stem cells in a subject comprising administering a cell of the invention to the subject under conditions sufficient for the cell to propagate, migrate, differentiate or a combination thereof, so as to provide a reservoir of neural stem cells to repair damaged neural stem cells in the subject, thereby, repairing damaged neural stem cells in the subject. In one embodiment, the neural stem cells of the invention provide growth factors to support neural stem cells, such as EGF, basic FGF, FGF 10, and/or Shh (Sonic Hedgehog). In one embodiment, the neural stem cells of the invention provide structural support for the repair of damaged neural stem cells in the subject.

Further, the invention provides a method of treating a neurodegenerative disease in a subject comprising administering a cell of the invention to the subject under conditions sufficient for the cell to propagate, migrate, differentiate or a combination thereof, so as to repair or replace lost, damaged or dying neural or glial cell and restore structure or function, thereby treating a neurodegenerative disease in the subject.

Examples of neurodegenerative disease include, but are not limited to, Parkinson's disease, Huntington's disease, multiple sclerosis, Alzheimer's disease, stroke, and traumatic brain injury.

Additionally, the invention provides a method of treating brain injury in a subject comprising administering a cell of the invention to the subject under conditions sufficient for the cell to propagate, migrate, differentiate or a combination thereof, so as to repair or replace lost, damaged or dying neural or glial cell and restore structure or function, thereby treating the brain injury in the subject. In one embodiment, the brain injury is traumatic brain injury.

The invention further provides a method of preventing apoptosis of neural cell, glial cell or a combination thereof associated with a loss of a neurotrophic factor in a subject comprising administering a cell of the invention into the subject under conditions sufficient for the cell to secrete the neurotrophic factor so as to increase its concentration around the neural cell, glial cell or both and prevent apoptosis, thereby preventing apoptosis of neural cell, glial cell or a combination thereof associated with the loss of a neurotrophic factor in the subject. Examples of neurotrophic factors include any of NGF, BDNF, GDNF, A and B ephrins, CTNF, and/or VEGF.

Further, the invention also provides a method of treating a tumor in a subject comprising administering a cell of the invention to the subject under conditions sufficient for the cell to migrate to the site of tumor so as to deliver a therapeutic agent, an enzyme, an antibody, a peptide, a small molecule or a combination thereof, so as to inhibit tumor growth, reduce tumor burden or eliminate the tumor, thereby treating the tumor in the subject. In one embodiment, the cells of the invention so administered expresses a therapeutic gene product, expresses an enzyme, or comprises an antibody, a peptide or a small molecule or a combination thereof.

In an embodiment of the invention, the subject may be administered additionally with a prodrug activating enzyme. For example, the prodrug activating enzyme is converting prodrug to become cytotoxic or cytostatic. Examples of the tumor may be a glioma, ependymoma or medulloblastoma.

The invention also provides method of delivering therapeutic agent to a site of brain damage or injury in a subject comprising administering the cell of the invention to the subject under conditions sufficient for the cell to migrate to the site of brain damage or injury, thereby delivering the therapeutic agent to the site of brain damage or injury in the subject.

In accordance with the practice of the invention, in one embodiment, the cells of the invention further comprises a therapeutic agent. Suitable examples of therapeutic agent include a neurotrophic factor, a drug, a peptide, an antibody, a secreted factor, and/or siRNA. Further, administration of the cells of the invention may be intranasal, intracranial, intraventricular, intrathecal and intravenous administration.

The invention also provides method of repairing damaged bone, cartilage, muscle or adipose tissue in a subject comprising administering a cell of the invention to a subject under conditions sufficient for the cell to propagate, migrate, differentiate or a combination thereof, so as to provide a reservoir of mesenchymal stem cells, osteoblasts, chondrocytes, myocytes, or adipocytes and neural cells to repair damaged bone, cartilage, muscle or adipose tissue in the subject, thereby, repairing damaged bone, cartilage, muscle or adipose tissue in the subject. In an embodiment of the invention, repair comprises replacement of damaged tissue or recovery of damaged tissue.

In accordance with the practice of the invention the cell may be a single cell suspension, is embedded in a solid matrix, or is in a cluster with other cells. Further, administration of the cell to the subject may be through any of oral, intravenous, intramuscular, intrathecal, subcutaneous, sublingual, buccal, rectal, vaginal, ocular, otic, nasal, intranasal, inhalation, nebulization, cutaneous, topical, systemic, transdermal, or local means.

Additionally, the invention also provides method of expanding the cell population of the invention comprising bioreactor, such as an automated Quantum bioreactor.

Further, the invention provides a method of replenishing neural cells in a subject comprising introducing a cell of the invention into the subject under conditions sufficient for the cell to propagate, migrate, differentiate or a combination thereof, so as to provide a reservoir of neural stem cells, neural progenitor cells and neural cells to replenish neural cells in a subject, thereby, replenishing neural cells in the subject.

### Kits of the Invention

According to another aspect of the invention, kits are provided. Kits according to the invention include package(s) comprising composition of the invention.

The phrase “package” means any vessel containing compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes (including pre-filled syringes), bottles, and any packaging material suitable for a selected formulation and intended mode of administration and treatment.

The kit can also contain items that are not contained within the package but are attached to the outside of the package, for example, pipettes.

Kits may optionally contain instructions for administering compositions of the present invention to a subject having a condition in need of treatment. Kits may also comprise instructions for approved uses of components of the composition herein by regulatory agencies, such as the United States Food and Drug Administration. Kits may optionally contain labeling or product inserts for the present compositions. The package(s) and/or any product insert(s) may themselves be approved by regulatory agencies. The kits can include compositions in the solid phase or in a liquid phase (such as buffers provided) in a package. The kits also can include buffers for preparing solutions for conducting the methods, and pipettes for transferring liquids from one container to another. The kit may optionally also contain one or more other compositions for use in combination therapies as described herein.

The following examples are presented to illustrate the present invention and to assist one of ordinary skill in making and using the same. The examples are not intended in any way to otherwise limit the scope of the invention.

### Example 1

### Materials and Methods

### Generation and Characterization of Primary NSC Cultures

Fetal brain from elective abortions was obtained as discarded tissue from Advanced Bioscience Resources, Inc. under a City of Hope Institutional Review Board approved protocol (City of Hope IRB #10079). All procedures which involved stem cell isolation and propagation were performed under approved protocols (City of Hope SCRO #11002, and IBC #11016). Human brain tissue (from fetuses of 10-14 weeks of gestation) was dissociated using 0.1% collagenase and hyaluronidase solution (collagenase 3000 U/ml, hyaluronidase 1000 U/ml in Hank's Buffered Salt Solution, HBSS), and isolated NSCs were cultured in serum-free NSC medium (RHB-A medium; Stem Cell Science, UK) supplemented with basic fibroblast growth factor (bFGF, 10 ng/ml), epidermal growth factor (EGF, 10 ng/ml), L-glutamine (2 mM; Invitrogen), Gem21 NeuroPlex Serum-Free Supplement (Gemini Bio-Products, #400-160) and penicillin-streptomycin (Mediatech, 30-002-CI). Growth factors (bFGF and EGF) were added every other day and media were completely changed every seven days. Cells were passaged by gentle trituration of neurospheres.

RNA and DNA were isolated from LM-NSC008 p2 and p12, NSC008 p 2 and p 9, and differentiated LM-NSC008 cells to characterize gene expression. LM-NSC008 cells were differentiated in vitro by culturing in DMEM medium supplemented with 1% FBS for 10 days as previously described (Nunes et al., 2003). Samples for karyotype determination were prepared for G-banding karyotyping analysis using standard cytogenetic methods at City of Hope Cytogenetics Core Facility.

Generation of NSC Lines that Stably Express the L-MYC Transgene

The L-MYC expression vector (pMXs-hu-L-Myc) was obtained from Addgene (Cambridge, Mass.) (Okita et al., 2013). The L-MYC gene from pMXs-hu-L-Myc was re-cloned into a retroviral expression vector (pCMV-LL-PGK-puro) containing the puromycin selection marker. Retrovirus carrying the L-MYC gene was used for transduction of human fetal derived NSCs at multiplicity of infection (MOI 2.5).

### Boyden Chamber Cell Migration Assay

In vitro chemotaxis assays were conducted using 24-well cell culture plates with polycarbonate inserts (8-μm pore size; Millipore) as described previously (Gutova et al., 2010). Independent t-test was performed to determine statistically significant difference between NSC008 and LM-NSC-008 migration to U87 conditioned media (P=0.0003).

### Cytotoxicity Assays

Detailed methodology is presented in Supplemental Experimental Procedures.

### Colony Formation Assay

Detailed methodology is presented in Supplemental Experimental Procedures.

### Iron Labeling of NSCs

Human NSCs (HB1.F3.CD and LM-NSC008) were labeled with Molday ION Rhodamine B prior to intranasal administration. LM-NSC008s and HB1.F3.CDs were visualized within brain tissue by Prussian blue staining using Accustain Iron Stain Kit (Sigma-Aldrich).

### Imunohistochemistry (IHC) and Immunocytochemistry (ICC)

Detailed methodology is presented in Supplemental Experimental Procedures.

### Flow Cytometry Analysis of Primary NSC008 and LM-NSC008 Cells

Detailed methodology is presented in Supplemental Experimental Procedures.

### Serial Section Reconstruction and Measurement

Reconstruction and measurement was performed using Reconstruct (version 1.1.0.1) (Fiala, 2005) software as described previously (Gutova et al., 2013). Briefly, 7-11 serial brain sections (10 μm each) separated by 200 μm were imported into the Reconstruct program and aligned manually. NSC quantification was performed using bright field Prussian blue images, and auto-tracing of the tumor was done on adjacent eGFP stained-images. From the number of NSCs on each section analyzed, the total number of cells per brain was calculated using spacing between stained sections and approximation of NSC numbers on unstained sections.

### Animal Studies of LM-NSC08 Cell Distributions

All animal studies were performed under approved City of Hope Institutional Animal Care and Use Committee protocols (IACUC 12025). Animal studies used NOD scid IL2Rgamma^{null} (NSG) mice, 8-12 weeks old regardless of sex. For distribution studies, orthotopic tumor xenografts were generated by injecting human U251.eGFP.ffluc glioma cells (1×10⁵ in 2 μl of serum-free DMEM) into the right frontal lobe of NSG mice (study day 1). LM-NSC008 cells (2×10⁵ in 3 μl of PBS) were injected contra-laterally to the tumor (n=6 mice) on day 7 of the study. Control mice (n=2) did not receive tumor injection, but did receive sham injection of PBS (to mimic injection of tumor cells) and NSCs on the opposite side of the brain. Mice were evaluated for clinical signs before and after tumor or NSC injections. Experimental animals were monitored daily for any debilitating signs secondary to tumor growth, including: labored breathing, weight loss (>20% body weight), scruffy coat, hunched posture, hypo- or hyperthermia, impaired ambulatory movement, and inability to remain upright. Tumors were confirmed by Xenogen imaging prior to NSC administration. All mice were euthanized on day 4 after NSC administration. Brains were removed without perfusion and processed for histological examination using hematoxylin and eosin (H&E) and eGFP immunostaining to detect tumors and tissue morphology. Prussian blue staining was done to identify Molday ION-labeled NSCs, as described previously (Addicott et al., 2011).

Traumatic brain injury was generated in adult (C57BL/6) Cdh23^{ahl} mice via impactor device as described previously (Brody et al., 2007). Briefly, mice were subjected to a single moderate left lateral (CCI) injury with craniotomy under isoflurane anesthesia. An electromagnetic impact device was used for generation of moderate contusion TBI using the following coordinates: Bregma coordinates-X axis, 1.2 mm left of midline; Y axis, 1.5 mm anterior to Lambda and 2 mm deep. After the probe was inserted into the brain (2 mm deep), the dwell time was 100 msec. Then the hole was covered with plastic skull cap and the skin was sutured. Sham TBI injury was performed on a control group of mice, which included craniotomy but no impactor stimulation, as described previously (Brody et al., 2007).

### Intranasal Administration of NSCs

LM-NSC008 and HB1.F3.CD cells were administered intranasally on days 9 and 11 after mild TBI. Mice received intranasal drops of either LM-NSC008 or HB1.NSC.CD cells (5×10⁵ cells per 12 μl/mouse into both nostrils divided equally (6 μl) with 2 min intervals pre-labeled with Molday ION Rhodamine-B (Addicott et al., 2011; Gutova et al., 2015). Mice were euthanized two weeks after the last NSC administration. Brain tissue was harvested and fixed in 4% PFA. Paraffin-embedded sections were prepared and Prussian blue staining was performed to visualize iron-labeled hNSCs. Prussian blue stained sections were used to quantify the number of hNSC cells in each brain for TBI and sham groups.

### In Vivo Tumorigenicity Studies

To determine the fate of NSCs in non-tumor bearing mouse brain, NSG mice were injected with 5×10⁵ NSCs (n=6) intracranial and were euthanized at 1, 4 and 12 weeks after injection, after which their brains were harvested. The brains were then isolated, fixed and stained (every 10^{th} section) with H&E (American Master Tech Modified Mayer's Hematoxylin and Eosin) KI-67 (Dako), TUNEL (Chemicon), human NESTIN (Millipore). Quantification of percentile KI-67 positive cells was performed using previously stained sections relative to the dose on LM-NSC008 cells injected.

mRNA Deep Sequencing with Illumina Hiseq2500

Detailed methodology is presented in Supplemental Experimental Procedures.

### RNA-Seq Data Analysis

Detailed methodology is presented in Supplemental Experimental Procedures. The records of RNA-Seq data have been approved and assigned GEO accession number (GSE84166).

### Whole Exome Sequencing for Somatic Mutations and Copy Number Variation Analysis

Detailed methodology is presented in Supplemental Experimental Procedures.

### Generation and Characterization of L-MYC Transduced NSC Clones

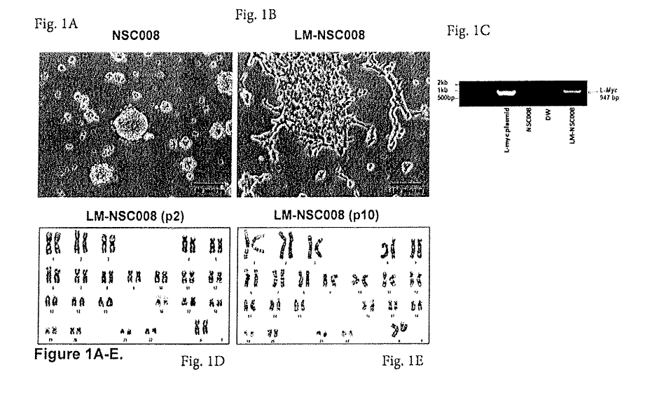

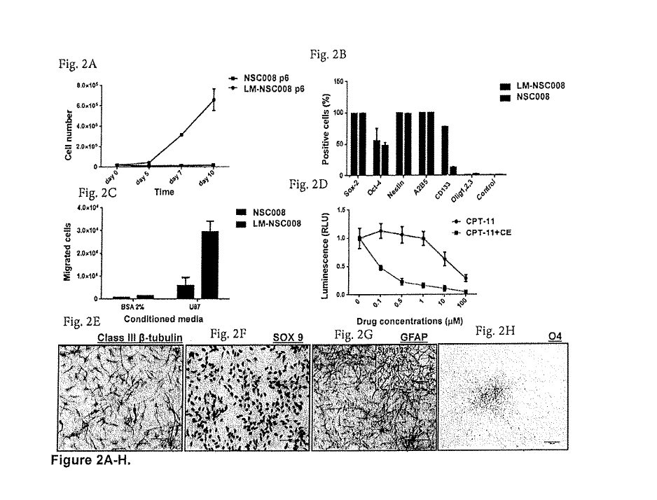

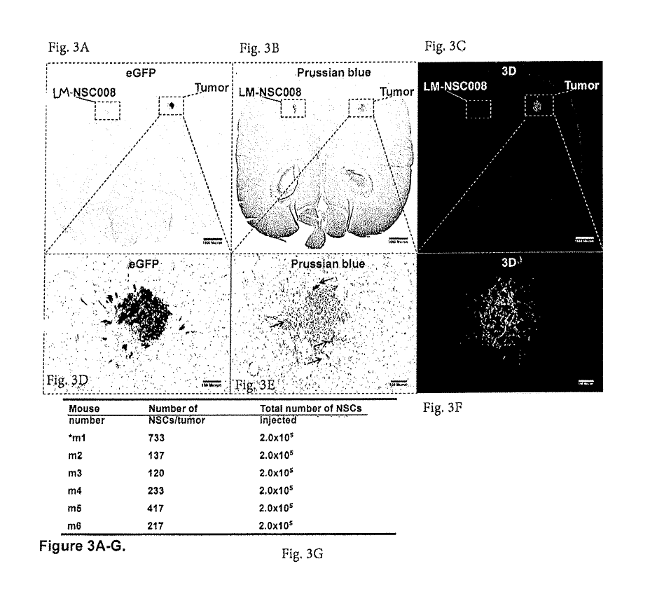

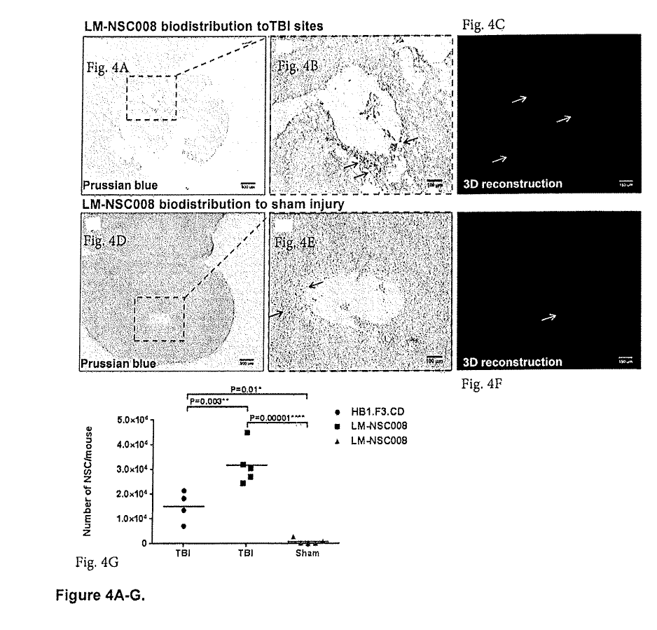

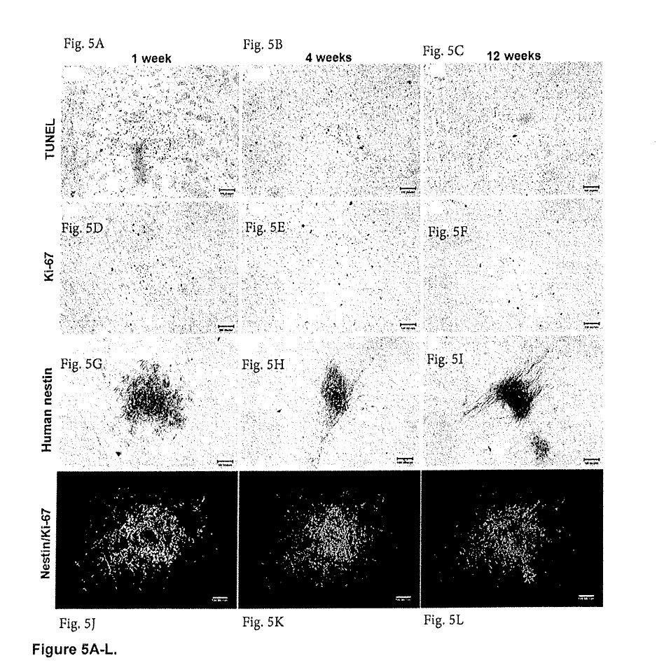

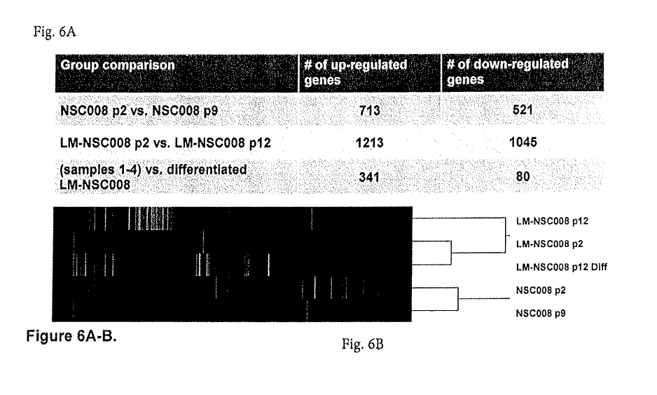

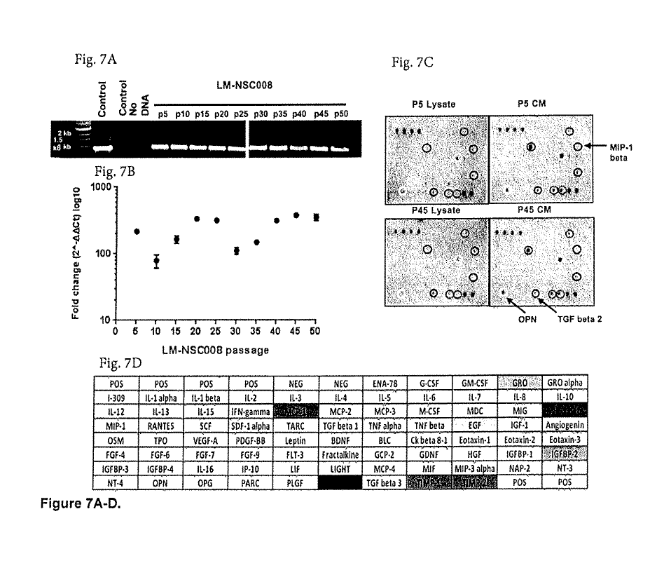

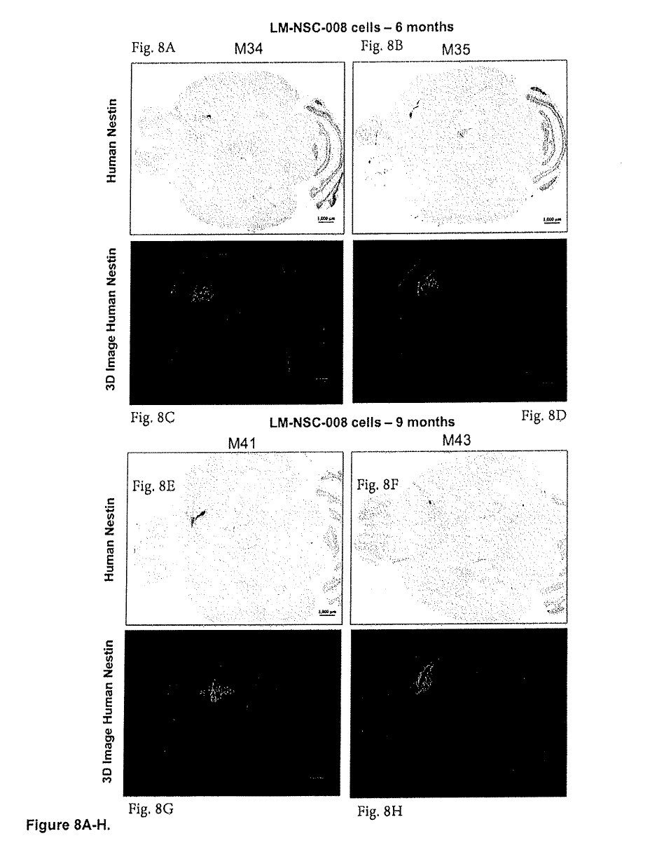

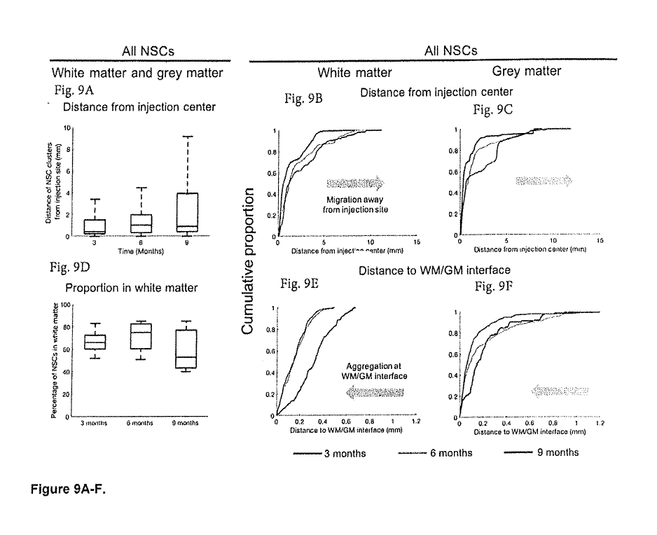

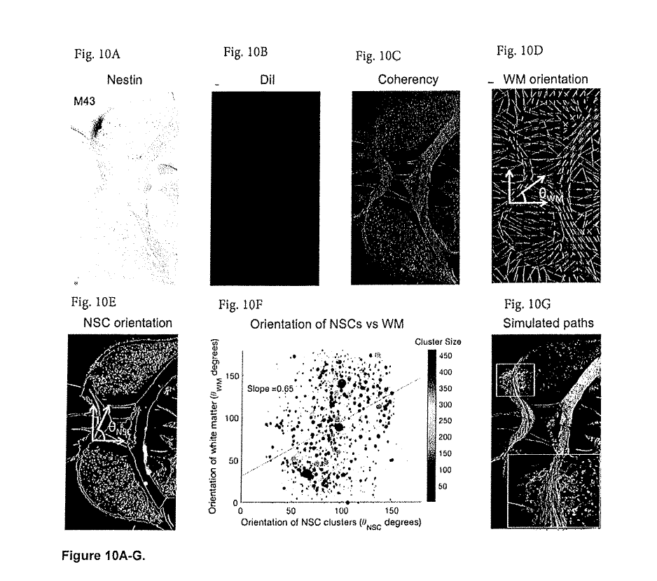

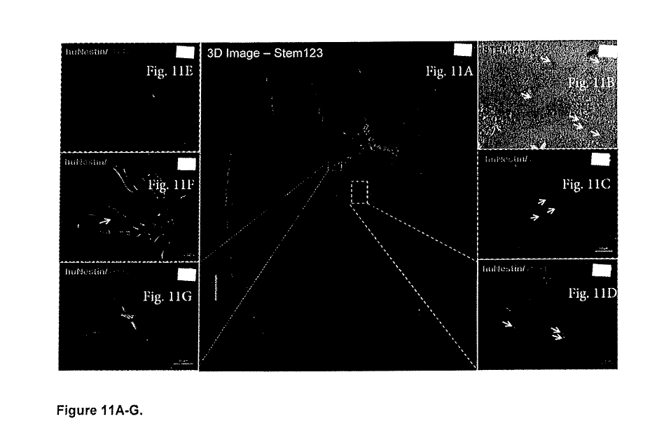

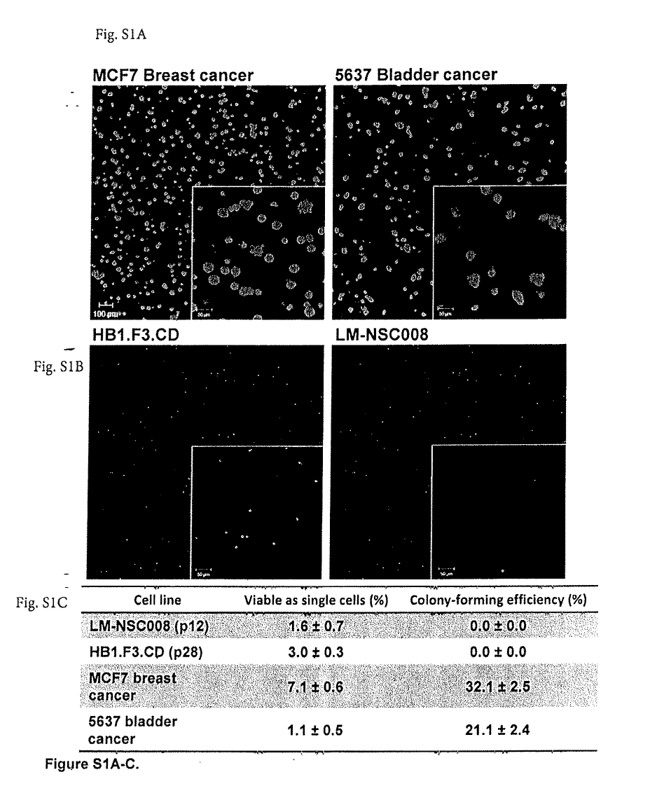

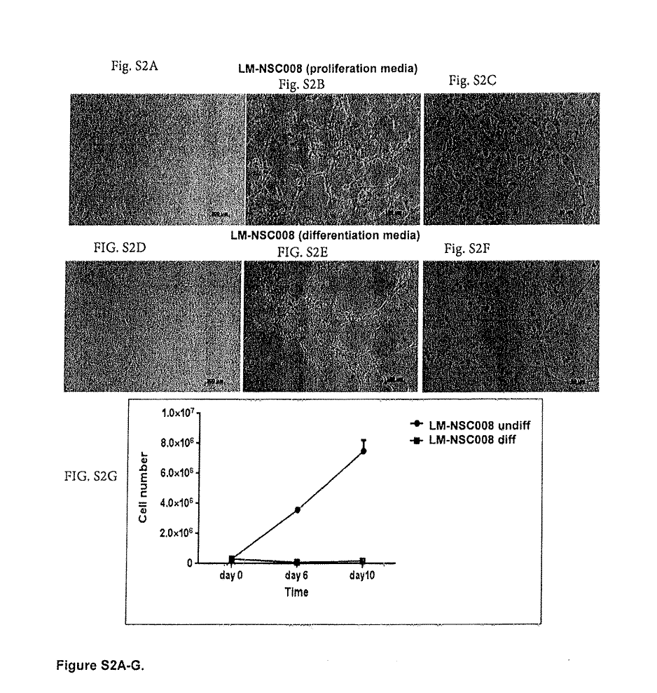

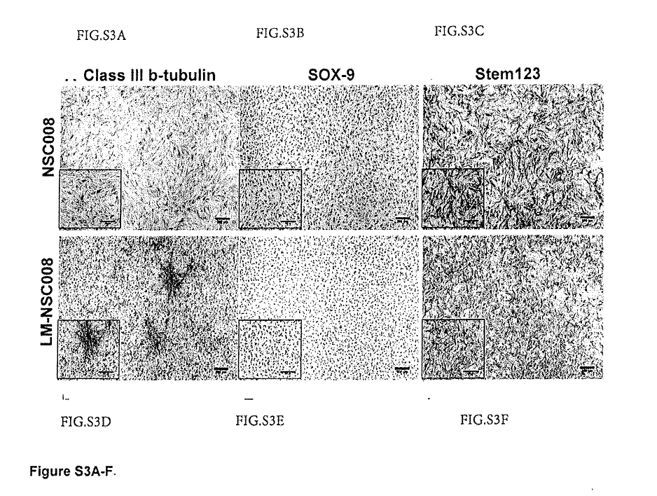

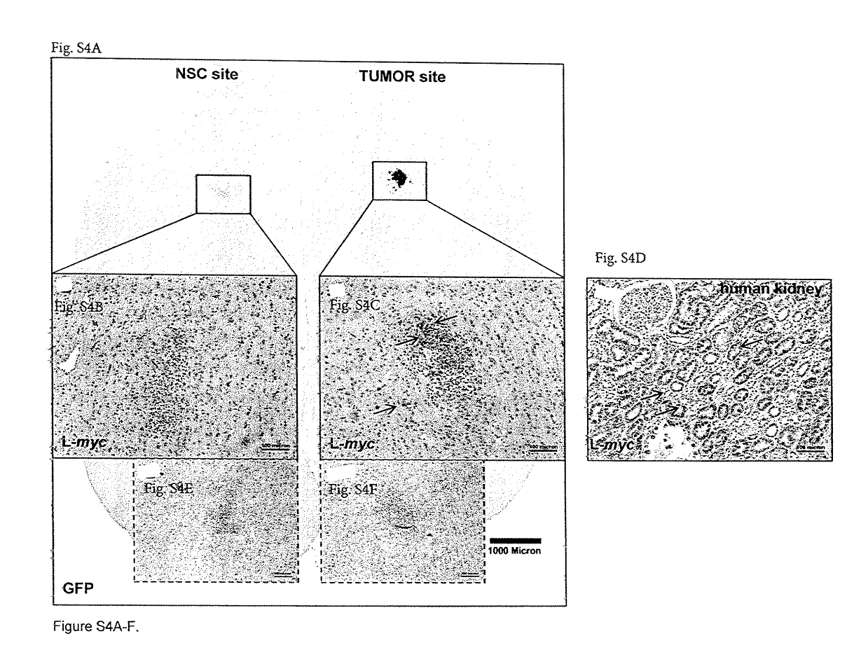

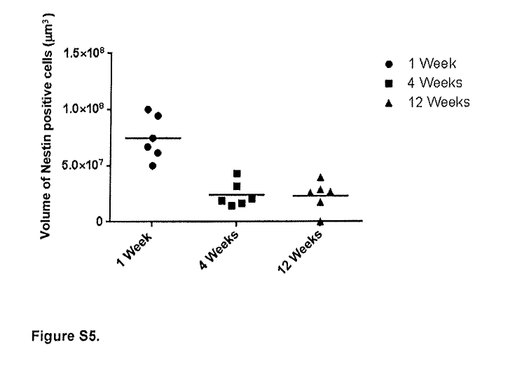

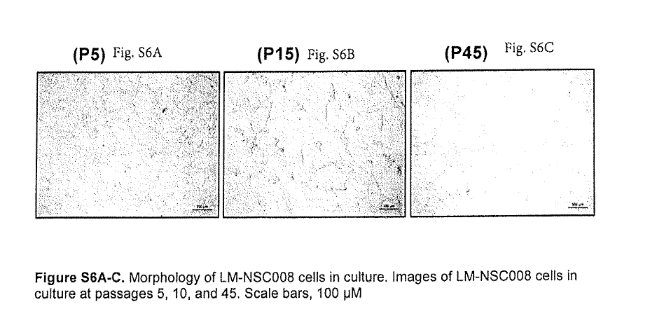

Cultures of dissociated NSCs were generated from human fetal brain tissue of 10-14 weeks gestation. NSCs were cultured under hypoxic conditions (4% 02) in a humidified incubator (Binder Inc.). In growth factor-supplemented stem cell medium, the primary hNSCs (NSC008) grew in suspension and formed neurospheres (FIG. 1A). At passage 2, we transduced the primary NSC008 cells with retrovirus carrying L-MYC and puromycin resistance gene (multiplicity of infection of 2.5). After 24-48 hours, transduced cells were grown in selection culture media containing puromycin (0.5 μg/ml), which eliminated all untransduced cells within 2-3 days. Cells were grown in puromycin for 28 days to select for NSCs stably expressing the L-MYC gene. The LM-NSC008 cells grown under the same culture conditions as primary NSC008s grew as a monolayer after 2-3 passages in vitro (FIG. 1B). This makes the LM-NSC008 cells more amenable to microscopic testing, cytology examination, and scale-up of cell production. Expression of the L-MYC gene was confirmed by genomic PCR analysis (FIG. 1C). LM-NSC008 cells had normal karyotype at passage 2 (p2) and p10 in vitro (FIG. 1D, E), and did not show abnormal growth or tumorigenicity in vitro (Fig. S1). Soft agar colony formation assays in 1% agarose hydrogels were performed to assess cellular anchorage-independent growth of L-MYC- (LM-NSC008) and v-MYC-transduced (HB1.F3.CD) hNSC cells in vitro as compared with MCF7 breast cancer and 5637 bladder cancer lines (Fig. S1). LM-NSC008 and HB1.F3.CD cells in contrast to the cancer cell lines (Fig. S1A), did not form colonies in soft agar, but rather remained viable as single cells (Fig. S1B). These observations are quantified in Fig. S1C.

### In Vitro Growth Kinetics, Tumor-Specific Migration and Multi-Lineage Differentiation of LM-NSC008 Cells

To determine whether the characteristics of these newly derived NSCs changed after expansion and passage in vitro, we compared primary NSC008 and LM-NSC008 grown through passage 6 with regard to viability, growth kinetics, and NSC marker expression (FIG. 2). Both transduced and untransduced cells showed >90% viability, grown in neurobasal medium under hypoxic conditions (4% 02). The growth kinetics of primary NSC008 and LM-NSC008 cells differed significantly when tested in culture over 10 days (using p6 NSC008 and LM-NSC008), showing significant differences in proliferation rate of NSC008 and LM-NSC008 cells (P=0.005 on day 5 and P=0.002 on days 7 and 10 as determined by multiple t-test analysis with Holm-Sidak adjustment for multiple comparisons) (FIG. 2A). The untransduced NSC008 did not proliferate (initial cell number at plating on day 1=2×10⁴/well, final cell number on day 10=1.8×10⁴) (FIG. 2A, blue line), whereas the LM-NSC008s showed robust proliferation (initial cell number at plating on day 1=2×10⁴, final cell number on day 10=6.6×10⁵; cell population doubling time ˜30 h for growth between days 5 and 10, after an initial lag time from day 0 to 5, red line). The untransduced NSC008 stopped dividing approximately at passages 6-10, while the LM-NSC008 cells continued to display robust growth even at passage p15 (the highest passage number we tested).

We used flow cytometry analysis and immunocytochemistry to detect stem cell and neural differentiation markers expressed by LM-NSC008 cells (FIG. 2B). Flow cytometry revealed that virtually all undifferentiated primary NSCs and LM-NSC008 cells expressed the neural stem cell markers SOX2 and NESTIN, fewer cells expressed Oct-4, and virtually no cells were Olig1, 2, 3 positive, consistent with a neural stem cell profile (FIG. 2B). LM-NSC008 and primary NSC008 differed in expression of the stem cell marker CD133 perhaps reflecting arrested development related to L-MYC expression in proliferation media (supplemented with FGF and EGF), LM-NSC008 cells divided approximately every 36 h, whereas they stopped dividing under pro-differentiation conditions as previously reported (Nunes et al., 2003) (Fig. S2G).

To explore the therapeutic potential of LM-NSC008 cells, we characterized their migration to tumor-conditioned media in vitro. In Boyden chamber cell migration assays, untransduced primary NSC008 at passage 6 exhibited little migration toward tumor cell-conditioned media as compared to LM-NSC008 cells at the same passage (FIG. 2C). These data suggest that after several passages in vitro, the untransduced parental NSC008 lack the abilities to proliferate and migrate toward tumor-conditioned media, whereas the LM-NSC008 cells possess proliferative and migratory properties through at least passage 15 (FIG. 2C). To demonstrate potential utility of LM-NSC008 cells in cancer targeted therapies, LM-NSC008 cells were adenovirally transduced to express a modified human CARBOXYLESTERASE (CE; hCElm6), which converts irinotecan (CPT-11) to SN-38, a potent topoisomerase I inhibitor (Wierdl et al., 2008; Metz et al., 2013). We performed in vitro cytotoxicity assays using human U251 glioma cells and various concentrations of irinotecan alone or in combination with CE derived from LM-NSC008 cells adenovirally transduced to express hCElm6. We observed enhanced irinotecan toxicity (500-fold) in the presence of hCElm6 in the culture media, when compared with CPT-11 alone. CE (hCElm6) activity was measured at 995 nmol/min/ml in the conditioned media derived from LM-NSC008 cells prior to cytotoxicity experiment (FIG. 2D). This suggests a potential use of LM-NSC008 cells for enzyme/prodrug therapy to treat glioma and other tumors.

To probe the differentiation potential of LM-NSC008 cells in vitro (FIG. 2E-H), we immunostained differentiated LM-NSC008 (grown for 10 days in differentiation media) for Class III β-TUBULIN, SOX9, GFAP and O4. The immunoreactivity observed indicated that LM-NSC008 cells were able to differentiate into neurons (Class III β-TUBULIN), astrocytes (SOX9, GFAP) and oligodendrocytes (O4) (FIG. 2E-H). We also compared differentiation of LM-NSC008 with untransduced primary NSC008 at passage 5 (Fig. S3). Differentiated LM-NSC008s and untrasduced NSC008 showed similar immunoreactivity for neuronal and glial markers Class III 3-TUBULIN, SOX9, and GFAP (Fig. S3).

Tumor-Directed Migration of hNSCs In Vivo

An orthotopic glioma xenograft model was generated by injecting U251.eGFP.ffluc human glioma cells (1×10⁵/2 μl PBS) into the right frontal hemisphere of adult NSG mice. Seven days later, iron-labeled LM-NSC008 cells (2×10⁵/3 μl PBS) were injected intracranially and contra-lateral to the tumor injection site (left frontal lobe, n=6 tumor-bearing mice, n=2 control non-tumor-bearing mice). Control mice received sham injections of PBS into the right frontal lobe, instead of tumor. Mice were euthanized 4 days after receiving LM-NSC008 injections, and their brains were harvested, fixed, sectioned, and stained for histological examination. Hematoxylin and eosin (H&E) and Prussian blue staining were used to visualize tumors and LM-NSC008 cells, respectively. Tumor sites were confirmed by immunostaining for eGFP (FIG. 3A, D with tumor area enlarged), which revealed the presence of compact tumor nodules located in the deep cortex and caudate-putamen, ranging in size between 0.6 to 1 mm diameters. Using Prussian blue staining, we could often identify the injection site of iron-labeled NSCs as a distinct, compact cellular focus located contra-lateral to the tumor site. Iron-labeled LM-NSC008s were found at the tumor site by 4 days post injection (FIG. 3B, E with tumor area enlarged) having migrated from contra-lateral hemisphere. LM-NSC008 cells were mostly found in peripheral areas of the tumor (FIG. 3E), dispersed within tumor nodules (FIG. 3F) and in proximity to infiltrating tumor cells. Three-dimensional (3D) reconstruction was used to quantify the number of NSCs at the tumor site and their spatial distribution within the tumor (FIG. 3C, F shows a Z-axis projection of 8-10 slices, 200 microns apart). Quantification of LM-NSC008 cell numbers at the tumor site is summarized in FIG. 3G. We have estimated the average number of LM-NSC008 cells within the tumor was 310±233, migrated from the opposite hemisphere.

Immunostaining for L-MYC confirmed its presence at the LM-NSC008 injection site and the tumor site in the contra-lateral hemisphere (Fig. S4A, enlarged in B, C). Human kidney tissue was used as a positive control for L-MYC staining (Fig. S4D). Negative control was obtained from omitted primary antibody staining (Fig. S4 E, F). LM-NSC008 injection site (E) and tumor site (F) shown.

### Migration of LM-NSC008 Cells to Sites of Traumatic Brain Injury

To show the potential of LM-NSC008 cells to target injured brain, we performed in vivo biodistribution experiments, using a controlled cortical impact traumatic brain injury (TBI) model. TBI was generated in adult C57BL/6 mice using an impactor device as described previously (Brody et al., 2007). Sham-operated mice (without TBI) were used as controls. The LM-NSC008 and HB1.F3.CD NSCs were administered intranasally on days 9 and 11 following generation of moderate TBI or a sham operation (n=5 per group), as described previously (Gutova et al., 2015). Mice received intranasal drops of hNSCs (5×10⁵ cells in 12 μl PBS, equally divided and administered into both nostrils. LM-NSC008 and HB1.F3.CD NSCs were pre-labeled with Molday ION Rhodamine-B as described previously (Addicott et al., 2011). Brain tissue was harvested 2-7 days after the last NSC administration, and iron-labeled hNSCs were visualized by Prussian blue staining. 3D reconstruction was performed using Prussian blue stained sections. LM-NSC008 cells showed robust migration to TBI injury sites (FIG. 4A, B), while sham-operated control mice had fewer LM-NSC008 stem cells at the surgery sites (FIG. 4D, E).

Numbers of LM-NSC008 and HB1.F3.CD hNSCs were quantified at TBI sites from Z-axis projections of Prussian blue-stained brain sections (FIG. 4C, F), and total numbers of hNSCs were calculated for each mouse. A summary is presented in FIG. 4G, comparing the results of: 1) LM-NSC008 cell migration to TBI and sham injury sites, P=0.00001****; 2) HB1.F3.CD migration to TBI and sham sites, P=0.01* (FIG. 4G). Comparison of TBI-directed migration of both hNSCs (LM-NSC008 and HB1.F3.CD) showed significant difference among TBI-specific migration of these lines, P=0.003** This data suggest that LM-NSC008 administered intranasally can reach sites of CNS injury in higher numbers than HB1.F3.CDs and have the potential to be used for brain tissue repair and/or regeneration.

### LM-NSC008 Fate in Normal Brain

To characterize the behavior of LM-NSC008 cells in normal brain (no tumor or TBI), we injected 5×10⁵ cells in 4 μl of PBS into the right frontal lobe of NSG mice (n=6) (FIG. 5). Mice were placed into 3 groups that were euthanized at 1, 4, or 12 weeks after LM-NSC008 injections. Brain sections were examined for the presence of viable LM-NSC008s, for the presence of markers indicating their fates (proliferation, differentiation or apoptosis). H&E staining of paraffin-embedded sections showed foci of architectural distortion associated with the presence of LM-NSC008 cells at the injection site (not shown). Immunohistochemical staining for human NESTIN confirmed the presence of LM-NSC008s at the injection sites and the minimal migration away from the site (FIG. 5G, H, I). TUNEL staining indicated apoptosis of LM-NSC008 cells in normal brain at 1, 4, 12 weeks (FIG. 5A, B, C). KI-67 staining (human-specific) indicated in vivo division of LM-NSC008s at 1, 4, and 12 weeks without formation of masses caused by uncontrolled LM-NSC008 proliferation (FIG. 5D, E, F). We did not observe neurological symptoms in mice that might be related to the injection of LM-NSC008 cells.

To further demonstrate the lack of tumorigenicity, numbers of KI-67 positive cells per mouse brain and their locations were determined from IHC-stained brain sections. Human NESTIN positive cells (pseudo-colored green) are highlighted and merged with KI-67 positive cells (red) in (FIG. 5J-L). The number of KI-67 positive cells for each mouse was compared to the total number of injected cells (5×10⁵/mouse), and their percentile presented in (FIG. 5M). Quantification of human NESTIN expression intensity is presented in Fig. S5 and demonstrated time-dependent decrease in NESTIN expression at the LM-NSC008 injection site.

### Gene Expression Profiling and Genome-Wide Analysis of Primary NSC008 and LM-NSC008 Cells

We performed RNA sequencing analysis to determine the transcriptional changes that occurred in the newly derived NSCs in vitro with and without expression of L-MYC. To examine early and late passages, we isolated RNA from primary untransduced NSC008 at passages 2 and 9 (hNSC p2 and hNSC p9, respectively) and from LM-NSC008 cells at passages 2 and 12 (LM-NSC008 p2 and LM-NSC008 p12, respectively). We also differentiated the LM-NSC008s in vitro, using a published protocol (Nunes et al., 2003), and isolated RNA for analysis (LM-NSC008 p12.Diff). We then performed RNA-Seq expression profiling on the samples. To probe for differential gene expression, we selected genes that showed either increased or decreased expression based on results using EdgeR (FIG. 6A), a Bioconductor package for differential expression (DE) analyses of RNA-Seq reads (Meng et al., 2014). We performed three group comparisons using DE gene selection criteria: (1) absolute fold change (FC)≧3 (for comparisons of hNSC p2 vs. p9 and LM-NSC008 p2 vs.p12); (2) absolute FC≧3 and p≦0.05 (for comparison of all previous samples with the LM-NSC008 p12 differentiated sample) (FIG. 6A). Hierarchical clustering analysis was performed using Cluster 3.0 and the average linkage clustering method (de Hoon et al., 2004). The results were visualized in Treeview and a heatmap was generated (Saldanha, 2004) (FIG. 6B).

All up- and down-regulated genes were further analyzed using DAVID functional annotation analysis to compare the same groups (LM-NSC008 p2 vs. LM-NSC008 p12) (Table 1). Further analysis compared hNSC008 p2 vs. hNSC008 p9 (Table S1) and LM-NSC008 at p2 and p12 were also compared with the LM-NSC008 differentiated sample (Table S2).

TABLE 1
DAVID Functional annotation analysis
LM-NSC008 p2 vs. LM-NSC008 p12

| | | | |
|---|---|---|---|
| | Fold | | |
| Term | enrichment | Count | P value |

Top 5 Enriched pathways upregulated

| | | | |
|---|---|---|---|
| Neuron differentiation | 3.154299 | 72 | 3.73E−18 |
| Transmission of nerve | 3.125009 | 57 | 3.55E−14 |
| impulse | | | |
| Synaptic transmission | 3.219573 | 50 | 5.28E−13 |
| Cell-cell signaling | 2.430563 | 76 | 8.98E−13 |
| Cell adhesion | 2.247814 | 82 | 6.21E−12 |

Top 5 Enriched pathways down regulated

| | | | |
|---|---|---|---|
| Cell adhesion | 2.77224 | 104 | 8.81E−22 |
| Biological adhesion | 2.768286 | 104 | 9.83E−22 |
| Regulation of cell | 4.447297 | 46 | 1.65E−17 |
| motion | | | |
| Cell motion | 2.906924 | 74 | 1.13E−16 |
| Actin cytoskeleton | 3.963039 | 48 | 4.85E−16 |
| organization | | | |

### Supplemental Experimental Procedures

### Cytotoxicity Assays

Human U251 glioma cell line was used in in vitro cytotoxicity assays as described previously (Metz et al., 2013). Briefly, cells were placed into 96-well plates (5,000 cells per well, in triplicate) in 100 ml of culture media per well and cultured for 24 hours. Irinotecan (CPT-11), diluted in control culture medium or conditioned medium from CE-expressing LM-NSC008 cells, was then added to final concentrations of 0-100 μM. Cells were incubated with irinotecan for 4 hours, after which medium was aspirated and replaced with drug-free fresh medium, and cells were grown for an additional 96 hours. Characterization of the toxic effect of CPT-11 and CPT-11 in combination with hCElm6 on U251 glioma cells was performed using the CellTiter-Glo® Luminescent Cell Viability Assay to measure ATP activity as an indicator of cell viability (Promega, G7571). Mean values±SD of three independent experiments in triplicate measurements are shown. Multiple t-test analysis (Prism Version 6) with Holm-Sidak adjustment for multiple comparisons was performed to analyze significant differences among CPT-11 and CPT-11+CE treated samples for each CPT-11 concentration (0-100 μM). P<0.0001 values for (0.5-100 μM).

### Colony Formation Assay

A standard soft agar colony formation assay was used to assess anchorage-independent NSC growth in vitro. Human tumor cells (5637 bladder and MCF7 breast, ATCC) and LM-NSC008 (p12), HB1.F3.CDs (p28) were encapsulated at 1×10⁵ cells/ml within 50 μl of 1% w/w agarose hydrogels cured in a 96-well plate. Gels were labeled with Calcein-AM (Life Technologies) and imaged using a confocal microscope (Zeiss Axial observer Z1) after culture for 7 days in complete growth media. ImageJ was used to count and measure sizes of cells and cell clusters present in Z-axis projections of 13 optical sections spaced 100 μm apart.

### Imunohistochemistry (IHC) and Immunocytochemistry (ICC)

Paraffin-embedded brain sections (10 μm) were processed for histological staining for H&E (hematoxylin eosin). Immunostaining was performed using anti eGFP (Abcam 290, 1:500 dilution), TUNEL (ApopTag® Peroxidase In Situ Apoptosis Detection Kit cat. #S7100, Millipore), anti Ki-67 (Chemicon, MAB4062, at 1:25 dilution), anti Human Nestin (Chemicon MAB5326 at 1:100 dilution) and anti L-myc (Santa Cruz Biotechnology, Inc. SC-28699, at dilution 1:100) antibodies. Immunoreactivity was visualized using Thermo Scientific Pierce DAB Substrate Kit which enables chromogenic detection of horseradish peroxidase (HRP) activity based the action of 3,3′-diaminobenzidine (DAB).

For cell differentiation experiments, primary human NSC008s and LM-NSC008 cells were cultured in DMEM supplemented with 1% fetal bovine serum (FBS). After 10 days, cells were fixed with 4% paraformaldehyde (PFA) for 4 min and washed in phosphate buffered saline (PBS) twice. Fixed cells were stained using standard ICC protocol using the following antibodies: 1) Class III β-Tubulin ((TUJ1) MMS435P, Covance Inc., at dilution 1:2000); 2) SOX9 (AF3075, R&D Systems Inc., at dilution 1:20); 3) GFAP (SMI-21R, Covance, at 1:1000 dilution); 4) human GFAP (Stem123, Y40420, Takara Bio Inc., at dilution 1:1000); 5) O4 (MAB1326, R&D Systems Inc. at dilution 1:100).

### Flow Cytometry Analysis of Primary NSC008 and LM-NSC008 Cells

Cells were harvested at passage 8, washed and incubated with antibodies in room temperature for 30 minutes using staining buffer (PBS+1% FBS). Unstained cells or cells stained with IgG isotype control antibodies were used as negative controls. Acquisition and analysis was performed on a Guava Express Plus 3.1 and software. Concentrations of antibodies used for flow cytometry were as follow: 1) SOX2 (FCMAB112F, Millipore Corp., at dilution 1:100); 2) OCT4 (FCMAB113A4, Millipore Corp., at dilution 1:100); 3) Nestin (IC1259F, R&D Systems, at dilution 1:20); 4) A2B5 (MAB1416, R&D Systems, 2.5 mg/10⁶ cells); 5) CD133 (130-080-801, Miltenyi Biotech, at dilution 1:11), and 6) Olig 1, 2, 3 (IC2230P, R&D Systems, at dilution 1:20). RNA-Seq data is provided to support flow cytometry results for expression level of SOX2, PAX6 and OCT4 genes (Table S3).

mRNA Deep Sequencing with Illumina Hiseq2500

Sequencing libraries were prepared with the TruSeq RNA Sample Preparation Kit V2 (Illumina) according to the manufacturer's protocol with minor modifications. Briefly, 500 ng of total RNA from each sample was used for polyadenylated RNA enrichment with oligo dT magnetic beads, and the poly (A) RNA was fragmented with divalent cations under elevated temperature. First-strand cDNA synthesis produced single-stranded DNA copies from the fragmented RNA by reverse transcription. After second-strand cDNA synthesis, the double-stranded DNA underwent end repair, and the 3′ ends were adenylated. Finally, universal adapters were ligated to the cDNA fragments, and 10 cycles of PCR were performed to produce the final sequencing library. Library templates were prepared for sequencing using the cBot cluster generation system (Illumina) with TruSeq SR Cluster V3 Kit.

Sequencing runs were performed in single read mode of 51 cycle of read 1 and 7 cycles of index read using Illumina HiSeq 2500 platform with TruSeq SBS V3 Kits. Real-time analysis software was used to process the image analysis and base calling. Sequencing runs generated approximately 40 million single reads for each sample.

### RNA-Seq Data Analysis

The 51-bp-long single-ended sequence reads were mapped to the human genome (hg19) using TopHat, and the frequency of Refseq genes was counted using customized R scripts. The raw counts were then normalized using the trimmed mean of M values (TMM) method and compared using Bioconductor package “edgeR” (Robinson et al., 2010). Reads per kilobase per million (RPKM) mapped reads were also calculated from the raw counts.

Differentially expressed genes were identified if RPKM≧1 in at least one sample, fold change≧3, and P≦0.05. Gene enrichments were analyzed using DAVID (National Institute of Allergy and Infectious Diseases (NIAID), 2016). Hierarchical clustering analysis was performed in cluster 3.0 using the average linkage clustering method (Wilson et al., 2004). The results were then visualized in Treeview and a heatmap was generated (Saldanha, 2004). Differentially expressed (DE) gene lists from the group comparison mentioned above were uploaded to DAVID v6.7 (Dennis et al., 2003) to identify enriched biological themes particular for GO terms and KEGG pathway (Tables S1, S2).

### Whole Exome Sequencing for Somatic Mutations and Copy Number Variation Analysis

Coding exons from genomic DNA samples were amplified using the Ion Ampliseq Exome kit (Catalog number 4487084, ThermoFisher Scientific Inc.). The amplified products were loaded onto the Proton Q1 chip and sequenced using Ion Proton sequencer following manufacturer's recommendations. For each sample, about 25 million reads were generated, which provided coverage of 52× for P2 cells and 64× for P12 cells for the coding exons. DNA sequences were aligned to hgl9 with Torrent Suite TMAP. Somatic mutations were identified using VarScan (Koboldt et al., 2012) and further filtered to remove false positives with the following criteria: 1) allele total coverage in P12>10; 2) alternative allele frequency in P12>=25%; 3) Alternative allele is not detectable in P2 sample. Copy number variation analysis was performed as described previously (Zhang et al., 2013). Briefly, exon level coverage for each coding exon in the RefSeq genes were counted using custom R scripts and Bioconductor package “ShortRead” and “Granges”. Copy number variation was detected using R package “DNAcopy” with log 2 fold change of exon level coverage between the two cell types LM-NSC008-p12 vs p2 (Table S4).

TABLE S1
DAVID Functional annotation analysis NSC008 p2 vs. NSC008 p9

| | | | |
|---|---|---|---|
| | Fold | | |
| Term | Enrichment | Count | P Value |

Top 5 Enriched Pathway upregulated

| | | | |
|---|---|---|---|
| Transmission of nerve impulse | 3.804615 | 44 | 7.21E−14 |
| Synaptic transmission | 3.960722 | 39 | 6.88E−13 |
| Regulation of nervous system | 4.571122 | 29 | 3.35E−11 |
| development | | | |
| Cell-cell signaling | 2.723758 | 54 | 4.73E−11 |
| Regulation of neurogenesis | 4.740142 | 26 | 1.92E−10 |

Top 5 Enriched Pathway down-regulated

| | | | |
|---|---|---|---|
| Immune response | 2.825541 | 49 | 1.08E−10 |
| Antigen processing and presentation | 14.46845 | 12 | 2.46E−10 |
| of peptide or polysaccharide antigen | | | |
| via MHC class II | | | |
| Skeletal system development | 3.866568 | 31 | 4.55E−10 |
| Cell adhesion | 2.728336 | 48 | 5.69E−10 |
| Biological adhesion | 2.724444 | 48 | 5.87E−10 |

TABLE S2
DAVID Functional annotation analysis of NSC008
and LM-NSC008 vs. differentiated LM-NSC008

| | | | |
|---|---|---|---|
| | Fold | | |
| Term | Enrichment | Count | P Value |

Top 5 Enriched Pathway upregulated

| | | | |
|---|---|---|---|
| Cell adhesion | 3.380163 | 46 | 8.39E−13 |
| Biological adhesion | 3.375341 | 46 | 8.70E−13 |
| Response to wounding | 3.590903 | 37 | 4.51E−11 |
| Regulation of cell growth | 5.302811 | 20 | 6.99E−09 |
| Regulation of growth | 3.921903 | 26 | 1.14E−08 |

Top 5 Enriched Pathway down-regulated

| | | | |
|---|---|---|---|
| Skeletal system development | 6.468944 | 9 | 6.02E−05 |
| Cellular di-, tri-valent inorganic | 6.060479 | 6 | 0.002831 |
| cation homeostasis | | | |
| Sensory organ development | 6.007549 | 6 | 0.00294 |
| Prostanoid metabolic process | 36.20339 | 3 | 0.002948 |
| Prostaglandin metabolic process | 36.20339 | 3 | 0.002948 |

TABLE S3
Gene expression level for SOX2, PAX6 and OCT4 genes.

| Gene | | | | | | | |
|---|---|---|---|---|---|---|---|
| Symbol | LogFC | Default. | LM-NSC008-p2 | LM-NSC008-p12 | NSC008-p2 | NSC008-p9 | LM-NSC008-p12-Diff |
| SOX2 | 0.519168 | False | 140.5328 | 84.29378 | 206.0346 | 147.9045 | 103.1067 |
| PAX6 | −0.303789 | False | 8.208481 | 5.112254 | 3.449178 | 4.380321 | 5.294839 |
| OCT4 | 6.007549 | False | 0.3 | 0.1 | 0.2 | 0.2 | 0.2 |
| (POU5F1) | | | | | | | |

TABLE S4
Copy Number Variation (CMV) detection analysis in LM-NSC008 cells

| | | | | | Number | | |
|---|---|---|---|---|---|---|---|
| ID | Chromosome | Start | End | Width | of Exons | Log2 FC | Gene |
| LM-NSC008-p12_vs_p2 | Chr14 | 50808850 | 50811704 | 2855 | 2 | −1.6103 | CDKL1 |
| LM-NSC008-p12_vs_p2 | Chr3 | 123367818 | 123368009 | 192 | 2 | −1.8286 | MYLK |

Comparison of gene expression in untransduced NSC008 at p2 vs. p9 revealed up-regulation of genes involved in pathways regulating neurogenesis, transmission of nerve impulses, synaptic transmission, regulation of nervous system development, and cell-cell signaling (Table S1). Genes involved in immune response and cell adhesion pathways were down-regulated (Table S1). Up-regulated genes in the LM-NSC008 cells included those involved in pathways responsible for regulating the development of the neural system, while down-regulated genes were involved in cell adhesion and regulation of cell migration (Table 1). When NSC008s and LM-NSC008 cells were compared with the differentiated LM-NSC008 cells, we observed up-regulation of regulators of cell growth and wound healing, while metabolic pathways were down-regulated indicating slower proliferation rates and differentiation of LM-NSC008 cells cultured under pro-differentiation conditions (Table S2).

Analyses of somatic mutations and copy number variations with whole exome sequencing were also performed to check for potential mutation events or changes in the karyotype as a result of L-MYC expression. Only 8 somatic mutations were identified when comparing LM-NSC008 p12 vs. LM-NSC008 p2. Of these 8 mutations, 6 were located in introns and 2 were synonymous, so none were considered functional and most likely represent germline mutations. We also did not observe any large copy number variations, with only two regions covering 2 coding exons of gene CDKL1 and MYLK showing possible loss of one allele in the p12 sample (Table S4). Overall, we concluded that there were no significant differences in the genomic sequences between the p12 and p2 samples, indicating that L-MYC gene expression did not contribute to increased genomic instability in LM-NSC008 cells.

### Discussion

In this study we have shown that expression of the L-MYC gene in human fetal brain-derived NSCs can promote self-renewal and multipotency in these cells. Untransduced NSCs from the same primary pool proliferate for only a limited number of passages in vitro before undergoing apoptosis. Our findings demonstrate that stable expression of the L-MYC gene in NSC008 promotes their survival and proliferation while preserving their migration and differentiation properties in vitro and in vivo. In this way, the LM-NSC008 cells will provide for GMP grade production as well as modification for therapeutic purposes. RNA and DNA sequencing analysis suggest that the L-MYC gene regulates distinct sets of target genes (Nakagawa et al., 2008; Nakagawa et al., 2010). Most likely the L-MYC gene acts to suppress expression of genes involved in differentiation, as was demonstrated previously (Nakagawa et al., 2010). At the same time, analysis of specific somatic mutations demonstrated that L-MYC expression in LM-NSC008 cells did not increase their genetic instability.

The LM-NSC008 cells also recapitulated important phenotypes of NSCs: after differentiation, the LM-NSC008 cells expressed neuronal Class III β-TUBULIN, astroglial markers SOX9 and GFAP and oligodendrocyte marker O4, which coincided with neuronal, astrocytic and oligodendroglial morphologies, respectively (FIG. 2E, F, G, H) (Wegner and Stolt, 2005; Sofroniew and Vinters, 2010). These are key characteristics that stem cells must display if they are to be used in cell replacement therapies for neurodegenerative diseases or brain injury (Lemmens and Steinberg, 2013; Tang et al., 2013).

The MYC genes orchestrate diverse cellular functions in the normal cells, including self-renewal and proliferation, morphological maturation, apoptosis, energy utilization, and multipotent differentiation (Conacci-Sorrell et al., 2014). Nakagawa et al. previously showed that L-MYC has a robust ability to reprogram cells for generation of iPSCs (Nakagawa et al., 2010). Our gene expression profiling data are in accord with the reported wide-ranging effects of L-MYC gene expression in hNSCs. The large number of upregulated genes (1213) and downregulated genes (1045) in LM-NSC008 p2 vs. LM-NSC008 p12 (FIG. 6) may reflect the complex protein interactome in which L-MYC participates directly or indirectly via its DNA-binding and transactivation domains (Tu et al., 2015). The robust changes in the gene expression profile also suggest that the introduction of the L-MYC gene into hNSCs may lead to epigenetic plasticity that could underlie the large-scale changes in gene expression (Arrowsmith et al., 2012; Watanabe et al., 2013).

Research on the ‘druggable epigenome’ and the use of small molecule drugs to break the epigenetic barrier by modulating the activity of enzymes acting on the epigenome (e.g. DNA methylases/demethylases, histone methyltrasferases/demethylases, acetyltransfreases/deacetylases, and RNA-interference) has led to advances in basic, as well as translational science, including clinical development (Arrowsmith et al., 2012). We surmise that in future research epigenome-modulating drugs may be applicable for optimization of the tumor-targeting and therapeutic properties of therapeutic NSCs, including the LM-NSC008 cells. Such studies could use LM-NSC008 cells deposited in a master cell bank, from which various subclones obtained by epigenetic reprogramming could be generated.

Additionally, analysis of the immunological parameters of the LM-NSC008 cells will be a crucial component of future preclinical studies using this cell line. Such immunological parameters could include the levels of MHC class I and class II gene expression (that could determine the immunological interactions between the transplanted NSCs and the host immune system), as well as expression profiling of various cytokine receptor genes in the LM-NSC008 cells which could underlie the signaling pathways responsible for the tumor tropism and targeting the nervous system affected by various pathological conditions.

In summary, we have generated a stable, expandable human NSC line (LM-NSC008) by overexpression of the L-MYC gene. We have characterized the LM-NSC008 cell line in vitro and in vivo. Our findings demonstrate the potential therapeutic utility of the LM-NSC008 cells for therapeutic drug delivery to CNS tumors, restorative therapies for TBI, and possibly other diseases of the CNS.

### Example 2

### Experimental Procedures

### Cell Culture

Generation and characterization of primary NSC, expression of L-MYC gene and propagation of LM-NSC008 cells was described previously (Li et al., 2016).

### In Vivo Animal Studies (6 Month and 9 Month)

All animal studies were performed under approved City of Hope Institutional Animal Care and Use Committee protocols (IACUC 12025). Animal studies used NOD scid IL2Rgammanull (NSG) mice, 8-12 weeks old male and female mice, as described previously (Li et al., 2016) To determine the long-term fate of NSCs in non-tumor bearing mouse brain, adult NSG mice of both sexes were injected with 5×10⁵ NSCs/4 ul of PBS (n=5) intracranial into right frontal lobe and were euthanized at 6 and 9 month time points after NSCs injection. The brains were then isolated, post fixed in 4% PFA. Upon fixation brains were rinsed with 1×PBS and were transferred to 70% EtOH solution for dehydration and embedment into paraffin blocks. The paraffin blocks were later sectioned into 10 μm sections and stained (every 10th section) with H&E (American Master Tech Modified Mayer's Hematoxylin and Eosin), anti-human Nestin antibody (Millipore; Cat#MAB5326) and DiI lipophilic tracer (Invitrogen; D282). Two brains (one-from each group) were immersed into OCT (Optimal Cutting Temperature Compound) over dry ice to be cryosectioned into 10 μm sections for staining with STEM123 antibody specific for human GFAP (Cellartis; Cat#Y40420).

### IHC and DiI Staining

The paraffin-embedded brain sections were deparaffinized in xylene and rehydrated with ethanol. The brain sections were then processed for antigen retrieval with Proteinase K (Dako ready-to-use Cat # S3020). The tissue sections were incubated in peroxidase quenching solution (0.3% H2O2 made in 100% methanol) and then in blocking solution (50% BlockAid, Invitrogen B10710; 50% Western Blocking Reagent, Roche Applied Sciences 11921673001; 1% Triton-100×). The slides were stained with primary antibody in blocking solution (1:200 dilution) and incubated overnight at 4 C followed by 1 hour incubation at room temperature with biotinylated secondary antibody (1:250 dilution, Vector BA-2001). The sections were washed, incubated in avidin-biotin complex (ABC) solution for 1 hour at room temperature and 5 min in 3, 3′-Diaminobenzidine (DAB) substrate solution containing 0.25% hydrogen peroxide. Then the brain sections were washed and mounted with Cytoseal 8 mounting media (Richard-Allan Scientific). The STEM123 staining was done according to the protocol described above on frozen sections fixed with 4% PFA. The STEM123 primary antibody (1:1000 dilution) and secondary biotinylated antibody were used for the staining. The OCT-immersed brain sections were fixed with 4% PFA, dehydrated through graded ethanol series, and stained with DiI lipophilic tracer for 15 min. After incubation, the tissue sections were washed, rehydrated through graded ethanol, and mounted with fluorescence-compatible mounting media.

Genomic DNA and qRT-PCR Analysis

LM-NSC008 cells were cultured and passaged in complete growth medium and harvested every 5th passage. Genomic DNA was isolated by DNeasy kit (Qiagen, Valencia, Calif.) and PCR was performed to amplify the L-MYC gene. The product was analyzed by electrophoresis on a 0.8% agarose gel and visualized by ethidium bromide staining. L-myc-pMXs plasmid was used as a positive control and NSC008 and no template as negative controls. Total RNA was isolated using TRIzol LS reagent (Invitrogen, Carlsbad, Calif.) according to manufacturer's protocol. 100 ng RNA was reverse transcribed into cDNA using iScript cDNA Synthesis Kit (Bio-Rad, Hercules, Calif.). Real-time PCR (qRT-PCR) was performed in 25 μl reactions using iQ SYBR Green Supermix Kit (Bio-Rad, Hercules, Calif.) and primer sequences (L-MYC: 5′AGAGGCAGTCTCTGGGTATT3′ (SEQ ID NO.: 1); 5′TGTGCTGATGGATGGAGATG3′ (SEQ ID NO.: 2)) on a Bio-Rad CFX96. Values obtained from RT-PCR were normalized to GAPDH housekeeping gene and the relative expression of L-MYC gene was calculated using 2-ΔΔCt method. NSC008 lacking L-MYC gene and L-MYC plasmid were used as negative and positive controls respectively. The area of L-MYC PCR bands were analyzed using Image J software. The pick areas of PCR bands were quantified and represented as percentage of the total size of measured area. Two-sample t-test was conducted to test groups mean difference of PCR values when there were two groups in comparison whereas one way ANOVA was carried out for multiple group comparison.

### Detection of Expressed and Secreted Proteins

Protein array was conducted using Cytokine Antibody Array V (RayBiotech, Norcross, Ga., http://www.raybiotech.com). LM-NSC008 cells (p5 and p45) were cultured under conditions described previously (Li et al., 2016) for 7 days. Culture media (CM) was collected, cells were pelleted and lysed with lysis buffer (20 mM TRIS, 150 mM NaCl, 2 mM EDTA, 0.5% Triton X-100). Antibody array membranes were exposed to 1 mL of CM and 1 mL of lysates, containing 500 μg of total protein in blocking buffer. Membranes were incubated at 4° C. overnight and chemiluminescence films were developed per manufacturers' recommendations.

### Computational Analysis of Tissue Anisotropy

Tissue anisotropy and orientation was quantified in DiI-stained ex vivo tissue using the OrientationJ plugin for FIJI (Schindelin et al., 2012) following methods outlined by Budde (Budde et al., 2011; Budde et al., 2012; Puspoki et al., 2016). OrientationJ uses Fourier transform analysis to compute eigenvalues and eigenvectors of a structure tensor at each pixel in the DiI image. The eigenvalues are combined to produce measures of anisotropy (coherence), directionality (orientation), and other quantities that have not been used in this analysis. Coherence (C) measures the degree to which there is a dominant eigenvalue of the structure tensor C=(λₘₐₓ−λₘᵢₙ)/(λₘₐₓ+λₘᵢₙ) (see supplement for illustration and discussion of these concepts, including Figure S2). Coherence takes values from 0-1, with a value of 0 corresponding to isotropic and a value of 1 to anisotropic. Orientation is the angle of the dominant eigenvector measured from the positive abscissa axis. To generate the orientation and coherence maps, a cubic spline gradient method with a Gaussian window of 1 pixel standard deviation was used in the OrientationJ Analysis module of the OrientationJ plugin. The orientation of the WM was compared to the orientation of NSCs with respect to each other. To calculate the orientation of NSCs the following procedure was employed. The center of each NSC cluster defined by the nestin- or STEM123-stained image was dilated by 200 pixels and eroded by 100 pixels to create coalesced regions of NSC clusters yielding an NSC density map. A second degree polynomial was fit to each of these coalesced regions and a line tangent to this polynomial and closest to each NSC cluster center was calculated. The angle of this line with the abscissa indicates the orientation of NSCs (θ_{NSC}) for that cluster.

### Results

### Stability of LM-NSC008 Cells During Long-Term In Vitro Expansion

LM-NSC008 cells were grown under hypoxic conditions (4% θ₂) in a humidified incubator as described previously¹¹ (Li et al., 2016). In growth factor-supplemented stem cell medium, LM-NSC008 cells grew as a monolayer and showed no changes in growth rate up to passage 50 in vitro. The genomic DNA was isolated and the presence of L-MYC in LM-NSC008 cells was confirmed by PCR analysis of genomic DNA at every fifth passage (p5-p50) (FIG. 7A). Films were scanned from two independent experiments and each PCR band area was analyzed using ImageJ software. The values for L-MYC band intensities. The values for L-MYC band intensities derived from each passage of LM-NSC008s (p5 to p50) were compared using one-way ANOVA and showed no statistical difference among the bands (p=0.058). mRNA was also isolated in every fifth passage from LM-NSC008 cells and analyzed for L-MYC gene expression levels in all passages using real time PCR (RT-PCR) and L-MYC specific primers (FIG. 7B, experiments run in triplicate). RT-PCR analysis showed overall higher levels of L-MYC expression in LM-NSC008 cells over 50 passages (as one group) in comparison to untransduced NSC008 cells (P-value<0.001 by one-way ANOVA) in vitro. The accumulated mRNA levels were 78- to 377-fold higher (mean/median of 241/264) that those of untransduced NSC008 cells (FIG. 7B). These results showed stability of L-MYC gene expression in LM-NSC008 cells in in vitro passages.

To assess the stability of protein secretion and expression by LM-NSC008 cells over passages in vitro, we performed protein array analyses using conditioned medium (CM) and cell lysates (derived from pelleted cells). LM-NSC008 cells were cultured at low (p5) and high (p45) passages, and after 5 days of growth CM was collected and cells were collected and pelleted. CM derived from low (p5) and high (p45) passages showed comparable cytokine secretion profiles (MCP-1, EGF, TIMP-1, TGF-beta 2, OPN, and IGFBP-2) (FIG. 7C, right panels). Similar results were observed in lysates derived from LM-NSC008 cells from low and high passages, suggesting the stability of protein expression in vitro (FIG. 7C, left panels). Morphology of LM-NSC008 cells in culture in passages 5, 10 and 45 is shown in Figure S6.

### Migration of LM-NSC008 Cells at 6 and 9 Months Post-Injection in Naïve Mouse Brain

To observe the changes in the distributions of LM-NSC008 cells over months following implantation, we employed an orthotopic human NSC xenograft model injecting LM-NSC008 cells (5×10⁵/2 μl PBS) into the right frontal hemispheres of adult male and female NOD scid IL2R gamma null (NSG) mice (n=6). Mice were euthanized 6 or 9 months after LM-NSC008 injection, and brains were harvested, fixed, sectioned, and stained for histological examination. The migration and distribution characteristics of NSCs in brain sections of 3 month mice (IHC using human antibodies for nestin and Stem123 to visualize LM-NSC008 cells) were described previously (Li et al., 2016) and were included in this study for computational analyses along with 6 and 9 month data. Hematoxylin and eosin (H&E) and immunohistochemistry (IHC for hu-nestin and glial stain STEM123-stained sections) staining were performed to visualize normal brain tissue and LM-NSC008s, respectively. Selected axial brain sections (adjacent to hu-nestin or STEM123) were stained with the lipophilic cyanine dye 1,10-dioctadecyl-3,3,3030-tetramethylindocarbocyanine perchlorate (DiI) to identify myelin associated with white matter tracts. DiI-stained sections were aligned with hu-nestin or STEM123-stained sections using Reconstruct software (version 1.1.0.1) (Harris, 1999) (FIG. 8).

LM-NSC008 cells were present at the NSC injection site and migrating through the corpus callosum (FIG. 8). Few NSCs distant from the injection center were found localized in the central corpus callosum by 3 months. Further accumulation of LM-NSC008 cells was evident in the central corpus callosum by 6 months post-implantation, and by 9 months an increased number and higher density of NSCs was evident within white matter distant from the injection site, including the anterior commissure (AC).

Three dimensional (3D) reconstruction of brain using STEM123 stained sections (FIG. 11A). IHC staining using STEM123 antibodies indicated that LM-NSC008 cells were partially differentiated into glial cells (FIG. 11A, B). We have also confirmed the LM-NSC008 cells expressing hu-nestin and neuronal marker TUJ1 (FIGS. 11C-G). Morphologically, we have identified neuron-like structures of LM-NSC008 cells expressing eGFP (FIGS. 11C-G). Here we address the successful engraftment, migration, in vivo differentiation, the lack of tumorigenicity of LM-NSC008 cells up to 9 month in naive mouse brain. The ability of LM-NSC008 cells to integrate/engraft into mouse brain, migrate away from injection site preferentially using white matter (WM) tracts and accumulate at white matter/grey matter (WM/GM) interfaces and fate of these cell in naïve brain, was further analyzed using computational prediction model.

### Computational Analysis of NSC Distribution and Migration

Migration of LM-NSC008 cells away from the injection site was quantified for the three time points: 3, 6 and 9 months (FIG. 9A). With time, LM-NSC008 cells were found to migrate away from the injection site. To quantify this migration we used 3, 6 and 9 month brain images (hu-nestin, STEM123 and DiI), and calculated the cumulative probability distribution (CDP) of distances from the injection site as a proportion of the total number of LM-NSC008 cells for each LM-NSC008 cluster. CDPs were calculated separately for the white (FIG. 9B) and grey matter (FIG. 9C). The survival and migration of LM-NSC008 cells in the brain can be inferred from the median distances of the NSC clusters from the injection site at 3, 6, and 9 months: 437 μm, 1030 μm, and 902 μm, respectively (combined white and grey matter data). Although the distances migrated at 6 and 9 months are comparable, it should be noted that those NSCs that at 9 months accumulated in the AC ipsilateral to the injection site did not have a linear path from the injection site and therefore likely traveled a longer distance than what was calculated. Thus, the total distance traveled by these NSCs was greater than the straight line distance from the injection site.

Since the LM-NSC008 cells were found migrating along the WM, we quantified NSC distribution by evaluating the percentage of NSCs in the WM and GM on the histological images. To quantify NSC distribution beyond the injection site, NSCs within a 1000 pixel (1444 μm) radius from the injection site were excluded from the spatial distribution analysis. Roughly 66%, 72%, and 59% of the NSCs were found within the WM (identified by regions of positive DiI staining) at the 3, 6, and 9 month time points, respectively (FIG. 9D).

We also observed that NSCs aggregated at WM/GM interfaces along the boundary of the AC. To quantify the degree to which NSCs aggregated along the WM/GM interfaces, we calculated the distance of LM-NSC008 cells to the closest WM/GM interface for each brain section and computed the CPDs for the NSCs in both WM (FIG. 9E) and GM (FIG. 9F). The 6-month and 9-month CPDs were lower than the 3-month time point, indicating an increased proportion of NSCs closer to WM/GM interfaces. At 6 months, NSC aggregation near the WM/GM interface was more evident for NSCs present in the WM as compared to 3 months post injection. The median distances of NSCs to the nearest WM/GM interface at 3, 6, and 9 months were 263 μm, 118 μm, and 87 μm, respectively (combined WM and GM data).

The alignment of NSCs relative to the tissue was evaluated by comparing the orientation of the LM-NSC008 cells relative to the orientation of the tissue. NSCs were identified by positive nestin or STEM123 staining (FIG. 10A). Firstly, tissue orientation was quantified on an adjacent section of DiI-stained tissue (FIG. 10B) using structure tensor analysis of these sections with the OrientationJ (Rezakhaniha et al., 2012) plugin for FIJI (FIG. 10C). The eigenvector associated with the largest eigenvalue of the structure tensor was used to calculate the dominant direction of the tissue at each pixel by calculating the angle of the principle eigenvector with respect to the abscissa axis (θ_{WM}) (FIG. 10D). Secondly, the orientation of NSCs (θ_{NSC}) (FIG. 10E) was calculated as described later and was positively correlated with the orientation of the DiI-defined white matter (θ_{WM}) (FIG. 10F; slope=0.65, r²=0.22). The linear regression was weighted by the total number of NSC clusters in each coalesced region analyzed (see experimental procedures section). This analysis, as well as visual inspection of the sections, suggests that NSCs migrate along WM and align their major axis with the structure of the brain tissue, specifically in this case with the WM.

Stochastic simulations were performed to simulate potential NSC migration paths by initializing 500 random seed points within a circular region in and around the corpus callosum (FIG. 10G). The orientation vectors at each point on the DiI image were calculated using OrientationJ with 5 pixel Gaussian kernel (to calculate stable θ_{WM}). These calculated θ_{WM} were used to simulate the NSC migration in the WM. In the GM, NSC migration angles were randomly sampled from a uniform distribution of 0-180 degrees relative to the direction of the migration vector of the previous time step. The paths were truncated if the NSC path intersected itself. Thus a predictive model of NSC migration in the brain, consistent with empirical observations was formulated.

### Discussion

LM-NSC008 cell line overcomes the major hurdles to the clinical translation of cell-based therapies by demonstrating long-term stability, lack of tumorigenicity and ease of being produced in sufficient quantities for clinical trials. Further corroborating their potential for use in clinical applications, LM-NSC008 cells demonstrated tropism to brain tumors and/or injury when administered IN or IC of LM-NSC008s in the mouse brain. These data support the use of LM-NSC008 cells for further therapeutic development of immortalized LM-NSC008 cells for allogeneic use in the treatment of brain tumors and injury.

In the absence of any pathological injury, NSCs were found to migrate along the corpus callosum (CC) when injected close to it. Migrating NSCs were localized to the CC (especially the central CC) by 3 months after injection (Li et al., 2016). Cross-sectional images of brains at 6 months post-injection, showed an increase in the number of NSCs distant from the injection site and a significantly increased number localized at the center of CC relative to the 3 month time point. Analysis of NSC distribution in brains 9 months post-injection showed NSCs clustered within the AC. These clustered NSCs aggregated at the interface of the WM/GM in the CC and AC. The aggregation at the WM/GM interface zones is consistent with a nonlinear anisotropic diffusion pattern predicted by mathematical models of cellular movement with differential rates of migration in the tissue (Belmonte-Beitia et al., 2013).

Our approach considered only NSC migration within the brain parenchyma, and did not account for leptomeningeal migration through the cerebrospinal fluid or via intravascular dispersion. Additionally, the analysis is a two-dimensional analysis of an inherently three-dimensional (3D) process. In our previous preclinical studies we used 3D reconstructions of serial tissue sections to analyze NSC distributions in relation to migration towards tumor targets. However, 3D serial reconstruction is time consuming and error prone, and interpretation of anatomical landmarks can be problematic. We therefore suggest 3D volumetric confocal microscopy of ex vivo sections over time as a potential technique for future studies to interrogate routes of NSC migration in the brain. The analyses and predictive computational model of NSC migration presented here are easily extended to three-dimensions.

The presented computational analysis of NSC migration and distribution provides proof-of-concept for the development of a more mechanistic computational model that can be used to predict NSC migration paths. Mathematical models of preferential migration along WM tracts have been used to predict distributions of malignant tumor cell, and may also be used to predict NSC migration routes (Adair et al., 2014; Trister et al., 2014; Painter and Hillen, 2015). We suggest that quantitative measures of tissue orientation and WM tracts determined from MR images (Gutova et al., 2013) can be used in a diffusion tensor imaging tractography-like approach to describe the most likely routes of NSC migration from origin to final target in a clinical setting. Such a model could be very useful in choosing the optimal location for NSC administration to a patient to achieve maximum therapeutic effect.

### REFERENCES FOR EXAMPLE 1

Aboody, K. S., Najbauer, J., Metz, M. Z., D'Apuzzo, M., Gutova, M., Annala, A. J., Synold, T. W., Couture, L. A., Blanchard, S., Moats, R. A., et al. (2013). Neural stem cell-mediated enzyme/prodrug therapy for glioma: preclinical studies. Sci Transl Med. 5, 184ra159.
Addicott, B., Willman, M., Rodriguez, J., Padgett, K., Han, D., Berman, D., Hare, J. M. and Kenyon, N. S. (2011). Mesenchymal stem cell labeling and in vitro MR characterization at 1.5 T of new SPIO contrast agent: Molday ION Rhodamine-B. Contrast Media Mol Imaging. 6, 7-18.
Arrowsmith, C. H., Bountra, C., Fish, P. V., Lee, K. and Schapira, M. (2012). Epigenetic protein families: a new frontier for drug discovery. Nat Rev Drug Discov. 11, 384-400.
Begum, A. N., Guoynes, C., Cho, J., Hao, J., Lutfy, K. and Hong, Y. (2015). Rapid generation of sub-type, region-specific neurons and neural networks from human pluripotent stem cell-derived neurospheres. Stem Cell Res. 15, 731-741.
Brody, D. L., Mac Donald, C., Kessens, C. C., Yuede, C., Parsadanian, M., Spinner, M., Kim, E., Schwetye, K. E., Holtzman, D. M. and Bayly, P. V. (2007). Electromagnetic controlled cortical impact device for precise, graded experimental traumatic brain injury. J Neurotrauma. 24, 657-673.
Conacci-Sorrell, M., McFerrin, L. and Eisenman, R. N. (2014). An overview of MYC and its interactome. Cold Spring Harb Perspect Med. 4, a014357.
Daniela, F., Vescovi, A. L. and Bottai, D. (2007). The stem cells as a potential treatment for neurodegeneration. Methods Mol Biol. 399, 199-213.
de Hoon, M. J., Imoto, S., Nolan, J. and Miyano, S. (2004). Open source clustering software. Bioinformatics. 20, 1453-1454.
Fiala, J. C. (2005). Reconstruct: a free editor for serial section microscopy. J Microsc. 218, 52-61. Gutova, M., Najbauer, J., Chen, M. Y., Potter, P. M., Kim, S. U. and Aboody, K. S. (2010).
Therapeutic targeting of melanoma cells using neural stem cells expressing carboxylesterase, a CPT-11 activating enzyme. Curr Stem Cell Res Ther. 5, 273-276.
Gutova, M., Shackleford, G. M., Khankaldyyan, V., Herrmann, K. A., Shi, X. H., Mittelholtz, K., Abramyants, Y., Blanchard, M. S., Kim, S. U., Annala, A. J., et al. (2013). Neural stem cell-mediated CE/CPT-11 enzyme/prodrug therapy in transgenic mouse model of intracerebellar medulloblastoma. Gene Ther. 20, 143-150.
Gutova, M., Shahmanyan, D., Oganesyan, D., Abramyants, Y., Danielyan, L., Frey II, W. H., Khankaldyyan, V., Najbauer, J., Balyasnikova, I. V., Moats, R. A., et al. (2015). Intranasal Delivery of Therapeutic Neural Stem Cells to Target Intracerebral Glioma. J Stem Cells Regen Med. 1, 007.
Hicks, C., Stevanato, L., Stroemer, R. P., Tang, E., Richardson, S. and Sinden, J. D. (2013). In vivo and in vitro characterization of the angiogenic effect of CTX0E03 human neural stem cells. Cell Transplant. 22, 1541-1552.

### SUPPLEMENTAL REFERENCES

Dennis, G., Jr., Sherman, B. T., Hosack, D. A., Yang, J., Gao, W., Lane, H. C. and Lempicki, R. A. (2003). DAVID: Database for Annotation, Visualization, and Integrated Discovery. Genome Biol. 4, P3.
Koboldt, D. C., Zhang, Q., Larson, D. E., Shen, D., McLellan, M. D., Lin, L., Miller, C. A., Mardis, E. R., Ding, L. and Wilson, R. K. (2012). VarScan 2: somatic mutation and copy number alteration discovery in cancer by exome sequencing. Genome Res. 22, 568-576.
Metz, M. Z., Gutova, M., Lacey, S. F., Abramyants, Y., Vo, T., Gilchrist, M., Tirughana, R., Ghoda, L. Y., Barish, M. E., Brown, C. E., et al. (2013). Neural stem cell-mediated delivery of irinotecan-activating carboxylesterases to glioma: implications for clinical use. Stem Cells Transl Med. 2, 983-992.
National Institute of Allergy and Infectious Diseases (NIAID). (2016). “Database for Annotation, Visualization, and Integrated Discovery (David), v 6.7.” Retrieved September 2015, from http://david.abcc.ncifcrf.gov.
Robinson, M. D., McCarthy, D. J. and Smyth, G. K. (2010). edgeR: a Bioconductor package for differential expression analysis of digital gene expression data. Bioinformatics. 26, 139-140.
Saldanha, A. J. (2004). Java Treeview—extensible visualization of microarray data. Bioinformatics. 20, 3246-3248. Wilson, L. L., Tran, L., Morton, D. L. and Hoon, D. S. (2004). Detection of differentially expressed proteins in early-stage melanoma patients using SELDI-TOF mass spectrometry. Ann. N. Y. Acad. Sci. 1022, 317-322.
Zhang, K., Chu, K., Wu, X., Gao, H., Wang, J., Yuan, Y. C., Loera, S., Ho, K., Wang, Y., Chow, W., et al. (2013). Amplification of FRS2 and activation of FGFR/FRS2 signaling pathway in high-grade liposarcoma. Cancer Res. 73, 1298-1307.
Kallos, M. S. and Behie, L. A. (1999). Inoculation and growth conditions for high-cell-density expansion of mammalian neural stem cells in suspension bioreactors. Biotechnol Bioeng. 63, 473-483.
Kanojia, D., Balyasnikova, I. V., Morshed, R. A., Frank, R. T., Yu, D., Zhang, L., Spencer, D. A., Kim, J. W., Han, Y., Yu, D., et al. (2015). Neural Stem Cells Secreting Anti-HER2 Antibody Improve Survival in a Preclinical Model of HER2 Overexpressing Breast Cancer Brain Metastases. Stem Cells. 33, 2985-2994.
Kim, S. U. (2004). Human neural stem cells genetically modified for brain repair in neurological disorders. Neuropathology. 24, 159-171.
Kim, S. U., Nagai, A., Nakagawa, E., Choi, H. B., Bang, J. H., Lee, H. J., Lee, M. A., Lee, Y. B. and Park, I. H. (2008). Production and characterization of immortal human neural stem cell line with multipotent differentiation property. Methods Mol Biol. 438, 103-121.
Lemmens, R. and Steinberg, G. K. (2013). Stem cell therapy for acute cerebral injury: what do we know and what will the future bring? Curr Opin Neurol. 26, 617-625. Meng, J., Lu, Z., Liu, H., Zhang, L., Zhang, S., Chen, Y., Rao, M. K. and Huang, Y. (2014). A protocol for RNA methylation differential analysis with MeRIP-Seq data and exomePeak R/Bioconductor package. Methods. 69, 274-281.
Metz, M. Z., Gutova, M., Lacey, S. F., Abramyants, Y., Vo, T., Gilchrist, M., Tirughana, R., Ghoda, L. Y., Barish, M. E., Brown, C. E., et al. (2013). Neural stem cell-mediated delivery of irinotecan-activating carboxylesterases to glioma: implications for clinical use. Stem Cells Transl Med. 2, 983-992.
Nakagawa, M., Koyanagi, M., Tanabe, K., Takahashi, K., Ichisaka, T., Aoi, T., Okita, K., Mochiduki, Y., Takizawa, N. and Yamanaka, S. (2008). Generation of induced pluripotent stem cells without Myc from mouse and human fibroblasts. Nat Biotechnol. 26, 101-106.
Nakagawa, M., Takizawa, N., Narita, M., Ichisaka, T. and Yamanaka, S. (2010). Promotion of direct reprogramming by transformation-deficient Myc. Proc Natl Acad Sci USA. 107, 14152-14157.
Nakagawa, M. and Yamanaka, S. (2010). Reprogramming of somatic cells to pluripotency. Adv Exp Med Biol. 695, 215-224.
Nunes, M. C., Roy, N. S., Keyoung, H. M., Goodman, R. R., McKhann, G., 2nd, Jiang, L., Kang, J., Nedergaard, M. and Goldman, S. A. (2003). Identification and isolation of multipotential neural progenitor cells from the subcortical white matter of the adult human brain. Nat Med. 9, 439-447.
Okita, K., Yamakawa, T., Matsumura, Y., Sato, Y., Amano, N., Watanabe, A., Goshima, N. and Yamanaka, S. (2013). An efficient nonviral method to generate integration-free human-induced pluripotent stem cells from cord blood and peripheral blood cells. Stem Cells. 31, 458-466.
Oster, S. K., Mao, D. Y., Kennedy, J. and Penn, L. Z. (2003). Functional analysis of the N-terminal domain of the Myc oncoprotein. Oncogene. 22, 1998-2010.
Pathan, S. A., Iqbal, Z., Zaidi, S. M., Talegaonkar, S., Vohra, D., Jain, G. K., Azeem, A., Jain, N., Lalani, J. R., Khar, R. K., et al. (2009). CNS drug delivery systems: novel approaches. Recent Pat. Drug Deliv. Formul. 3, 71-89.
Pollock, K., Stroemer, P., Patel, S., Stevanato, L., Hope, A., Miljan, E., Dong, Z., Hodges, H., Price, J. and Sinden, J. D. (2006). A conditionally immortal clonal stem cell line from human cortical neuroepithelium for the treatment of ischemic stroke. Exp Neurol. 199, 144-155.
Reynolds, B. A. and Weiss, S. (1992). Generation of neurons and astrocytes from isolated cells of the adult mammalian central nervous system. Science. 255, 1707-1710.
Saldanha, A. J. (2004). Java Treeview--extensible visualization of microarray data. Bioinformatics. 20, 3246-3248.
Sofroniew, M. V. and Vinters, H. V. (2010). Astrocytes: biology and pathology. Acta Neuropathol. 119, 7-35.
Stroemer, P., Patel, S., Hope, A., Oliveira, C., Pollock, K. and Sinden, J. (2009). The neural stem cell line CTX0E03 promotes behavioral recovery and endogenous neurogenesis after experimental stroke in a dose-dependent fashion. Neurorehabil Neural Repair. 23, 895-909.
Tang, H., Sha, H., Sun, H., Wu, X., Xie, L., Wang, P., Xu, C., Larsen, C., Zhang, H. L., Gong, Y., et al. (2013). Tracking induced pluripotent stem cells-derived neural stem cells in the central nervous system of rats and monkeys. Cell Reprogram. 15, 435-442.
Tu, W. B., Helander, S., Pilstal, R., Hickman, K. A., Lourenco, C., Jurisica, I., Raught, B., Wallner, B., Sunnerhagen, M. and Penn, L. Z. (2015). Myc and its interactors take shape. Biochim Biophys Acta. 1849, 469-483.
Umeda, K., Saida, S., Yamaguchi, H., Okamoto, S., Okamoto, T., Kato, I., Hiramatsu, H., Imai, T., Kodaira, T., Heike, T., et al. (2016). Central nervous system recurrence of desmoplastic small round cell tumor following aggressive multimodal therapy: A case report. Oncol Lett. 11, 856-860.
Watanabe, A., Yamada, Y. and Yamanaka, S. (2013). Epigenetic regulation in pluripotent stem cells: a key to breaking the epigenetic barrier. Philos Trans R Soc Lond B Biol Sci. 368, 20120292.
Wegner, M. and Stolt, C. C. (2005). From stem cells to neurons and glia: a Soxist's view of neural development. Trends Neurosci. 28, 583-588.
Wierdl, M., Tsurkan, L., Hyatt, J. L., Edwards, C. C., Hatfield, M. J., Morton, C. L., Houghton, P. J., Danks, M. K., Redinbo, M. R. and Potter, P. M. (2008). An improved human carboxylesterase for enzyme/prodrug therapy with CPT-11. Cancer Gene Ther. 15, 183-192.

### REFERENCES FOR EXAMPLE 2

Adair, J. E., Johnston, S. K., Mrugala, M. M., Beard, B. C., Guyman, L. A., Baldock, A. L., Bridge, C. A., Hawkins-Daarud, A., Gori, J. L., Born, D. E., et al. (2014). Gene therapy enhances chemotherapy tolerance and efficacy in glioblastoma patients. J Clin Invest. 124, 4082-4092.
Belmonte-Beitia, J., Woolley, T. E., Scott, J. G., Maini, P. K. and Gaffney, E. A. (2013). Modelling biological invasions: Individual to population scales at interfaces. J Theor Biol. 334, 1-12.
Budde, M. D., Gold, E., Jordan, E. K. and Frank, J. A. (2012). Differential microstructure and physiology of brain and bone metastases in a rat breast cancer model by diffusion and dynamic contrast enhanced MRI. Clin Exp Metastasis. 29, 51-62.
Budde, M. D., Janes, L., Gold, E., Turtzo, L. C. and Frank, J. A. (2011). The contribution of gliosis to diffusion tensor anisotropy and tractography following traumatic brain injury: validation in the rat using Fourier analysis of stained tissue sections. Brain. 134, 2248-2260.
Gutova, M., Frank, J. A., D'Apuzzo, M., Khankaldyyan, V., Gilchrist, M. M., Annala, A. J., Metz, M. Z., Abramyants, Y., Herrmann, K. A., Ghoda, L. Y., et al. (2013). Magnetic Resonance Imaging Tracking of Ferumoxytol-Labeled Human Neural Stem Cells: Studies Leading to Clinical Use. Stem Cells Transl Med. 2, 766-775.
Harris, K. (1999). SynapseWeb is a portal into the 3D ultrastructure of the brain. SynapseWeb.
Li, Z., Oganesyan, D., Mooney, R., Rong, X., Christensen, M. J., Shahmanyan, D., Perrigue, P. M., Benetatos, J., Tsaturyan, L., Aramburo, S., et al. (2016). L-MYC Expression Maintains Self-Renewal and Prolongs Multipotency of Primary Human Neural Stem Cells. Stem Cell Reports. 7, 483-495.
Painter, K. J. and Hillen, T. (2015). Navigating the flow: individual and continuum models for homing in flowing environments. J R Soc Interface. 12.
Puspoki, Z., Storath, M., Sage, D. and Unser, M. (2016). Transforms and Operators for Directional Bioimage Analysis: A Survey. Adv Anat Embryol Cell Biol. 219, 69-93.
Rezakhaniha, R., Agianniotis, A., Schrauwen, J. T., Griffa, A., Sage, D., Bouten, C. V., van de Vosse, F. N., Unser, M. and Stergiopulos, N. (2012). Experimental investigation of collagen waviness and orientation in the arterial adventitia using confocal laser scanning microscopy. Biomech Model Mechanobiol. 11, 461-473.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods. 9, 676-682.
Trister, A., Scott, J., Rockne, R., Yagle, K., Johnston, S. K., Hawkins-Daarud, A., Baldock, A. and Swanson, K. R. (2014). Response to “Tumor cells in search for glutamate: an alternative explanation for increased invasiveness of IDH1 mutant gliomas”. Neuro Oncol. 16, 1670-1671.

1. An isolated neural or mesenchymal stem cell population which is genetically modified with an exogenous nucleic acid sequence encoding for a L-MYC gene or its variant, wherein the L-MYC gene or its variant is necessary and sufficient to immortalize the isolated neural or mesenchymal stem cell population and wherein the isolated neural or mesenchymal stem cell population is not derived from an induced pluripotent stem cell population.

2. The isolated neural or mesenchymal stem cell population of claim 1, wherein expression of the exogenous nucleic acid sequence encoding for a L-MYC gene or its variant confers immortalization of an isolated neural or mesenchymal stem cell population.

3. (canceled)

4. (canceled)

5. (canceled)

6. The isolated neural or mesenchymal stem cell population of claim 3, wherein increased self-renewal and maintenance of multipotency of a neural or mesenchymal stem cell population is up to 50 cell passages.

7. (canceled)

8. The isolated neural or mesenchymal stem cell population of claim 1, wherein the stem cell population is genetically modified only with the L-myc gene.

9. The isolated neural or mesenchymal stem cell population of claim 1, wherein the cell population is free of genetic modification with an exogenous nucleic acid sequence encoding for a member of Myc transcription factor family other than L-myc or its variant, an Octomer-binding protein (Oct) transcription factor, a Sox transcription factor, a Klf transcription factor, a Nanog transcription factor, a LIN28 RNA-binding protein, a Glis1 transcription factor, a short hairpin RNA targeting p53, or a combination thereof.

10. (canceled)

11. (canceled)

12. (canceled)

13. (canceled)

14. (canceled)

15. The isolated neural or mesenchymal stem cell population of claim 1, wherein greater than about 90% of the neural stem cell population is positive for SOX2 and NESTIN neural stem cell markers.

16. (canceled)

17. (canceled)

18. (canceled)

19. (canceled)

20. (canceled)

21. (canceled)

22. (canceled)

23. (canceled)

24. (canceled)

25. The isolated neural or mesenchymal stem cell population of claim 1, wherein the neural or mesenchymal stem cell population is differentiated in vitro.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The isolated neural or mesenchymal stem cell population of claim 1 wherein the mammal is selected from the group consisting of a mouse, a rat, a rabbit, a cat, a dog, a bovine, a goat, a pig, a horse, a sheep, a monkey, a chimpanzee, and a human.

38. (canceled)

39. (canceled)

40. (canceled)

41. A defined cell population comprising a plurality of the cells of claim 1.

42. (canceled)

43. (canceled)

44. (canceled)

45. A progeny cell of the isolated neural or mesenchymal stem cell population of claim 1 committed to develop into a neural cell or a neuronal cell.

46. (canceled)

47. A method of producing an expandable neural or mesenchymal stem cell line comprising:
a. isolating neural or mesenchymal stem cells from a subject;
b. introducing an exogenous nucleic acid sequence encoding for a L-MYC gene or its variant into the isolated neural or mesenchymal stem cells of (a), wherein the L-MYC gene or its variant is necessary and sufficient to immortalize the isolated neural or mesenchymal stem cells so as to permit at least 50 passages without differentiating; and
c. culturing the cells of (b) under a condition permissive for self-renewal of neural or mesenchymal stem cells;
thereby, producing the expandable neural or mesenchymal stem cell line.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A method of replenishing lost neural cells or repairing damaged neural cells in a subject comprising administering a cell of claim 1, to the subject under conditions sufficient for the cell to propagate, migrate, differentiate or a combination thereof, so as to provide a reservoir of neural stem cells, neural progenitor cells and neural cells to replace lost neural cells or repair damaged neural cells in the subject, thereby, replenishing lost neural cells or repairing damaged neural cells in the subject.

67. A method of replenishing lost glial cells or repairing damaged glial cells in a subject comprising administering a cell of claim 1, to the subject under conditions sufficient for the cell to propagate, migrate, differentiate or a combination thereof, so as to provide a reservoir of neural stem cells, glial progenitor cells, astrocytes and oligodendrocytes to replace lost glial cells or repair damaged glial cells in the subject, thereby, replenishing lost glial cells or repairing damaged glial cells in the subject.

68. A method of treating a neurodegenerative disease in a subject comprising administering a cell of claim 1 to the subject under conditions sufficient for the cell to propagate, migrate, differentiate or a combination thereof, so as to repair or replace lost, damaged or dying neural or glial cell and restore structure or function, thereby treating a neurodegenerative disease in the subject.

69. The method of claim 68, wherein the neurodegenerative disease is selected from the group consisting of Parkinson's disease, Huntington's disease, multiple sclerosis, Alzheimer's disease, stroke, and brain injury.

70. A method of treating brain injury in a subject comprising administering a cell of claim 1 to the subject under conditions sufficient for the cell to propagate, migrate, differentiate or a combination thereof, so as to repair or replace lost, damaged or dying neural or glial cell and restore structure or function, thereby treating the brain injury in the subject.

71. (canceled)

72. A method of preventing apoptosis of neural cell, glial cell or a combination thereof associated with a loss of a neurotrophic factor in a subject comprising administering a cell of claim 1 into the subject under conditions sufficient for the cell to secrete the neurotrophic factor so as to increase its concentration around the neural cell, glial cell or both and prevent apoptosis, thereby preventing apoptosis of neural cell, glial cell or a combination thereof associated with the loss of a neurotrophic factor in the subject.

73. (canceled)

74. A method of treating a tumor in a subject comprising administering a cell of claim 1, to the subject under conditions sufficient for the cell to migrate to the site of tumor so as to deliver a therapeutic agent, an enzyme, an antibody, a peptide, a small molecule or a combination thereof, so as to inhibit tumor growth, reduce tumor burden or eliminate the tumor, thereby treating the tumor in the subject.

75. (canceled)

76. (canceled)

77. (canceled)

78. (canceled)

79. (canceled)

80. (canceled)

81. (canceled)

82. (canceled)

83. A progeny cell of the isolated neural or mesenchymal stem cell population of claim 1 committed to develop into a bone cell.

84. (canceled)

85. (canceled)

86. (canceled)

87. A method of repairing damaged bone, cartilage, muscle or adipose tissue in a subject comprising administering a cell of claim 1 to a subject under conditions sufficient for the cell to propagate, migrate, differentiate or a combination thereof, so as to provide a reservoir of mesenchymal stem cells, osteoblasts, chondrocytes, myocytes, or adipocytes and neural cells to repair damaged bone, cartilage, muscle or adipose tissue in the subject, thereby, repairing damaged bone, cartilage, muscle or adipose tissue in the subject.

88. (canceled)

89. (canceled)

90. (canceled)

91. (canceled)


SEQUENCE LISTING (2 sequences)

SEQ ID NO: 1 - DNA, 20 bp, Homo sapiens
  misc_feature (1)..(20): L-MYC Primer 1
agaggcagtctctgggtatt

SEQ ID NO: 2 - DNA, 20 bp, Artificial Sequence
  Part of Homo sapien L-MYC sequence with TG dinucleotide at 5′ end
  misc_feature (1)..(2): TG dinucleotide
  misc_feature (3)..(20): Homo sapien L-MYC sequence
tgtgctgatggatggagatg